(19) United States
(12) Patent Application Publication
(10) Pub. No.: US 2023/0172915 A1
(43) Pub. Date: Jun. 8, 2023

(54) DHODH INHIBITOR FOR THE TREATMENT OF COVID-19

(71) Applicant: PTC THERAPEUTICS, INC., South Plainfield, NJ (US)

(72) Inventors: Nikolai A. NARYSHKIN, East Brunswick, NJ (US); Jason GRACI, West Orange, NJ (US); John BAIRD, Bridgewater, NJ (US); Joseph COLACINO, Bayside, WI (US); Marla L. WEETALL, Morristown, NJ (US); Liangxian CAO, Watchung, NJ (US)

(73) Assignee: PTC THERAPEUTICS, INC., South Plainfield, NJ (US)

(21) Appl. No.: 17/923,825

(22) PCT Filed: May 7, 2021
(86) PCT No.: PCT/US2021/031326

§ 371 Date: Nov. 7, 2022

Related U.S. Application Data
(60) Provisional application No. 63/039,981, filed on Jun. 16, 2020.
(60) Provisional application No. 63/021,687, filed on May 8, 2020.

(51) Int. Cl. A61K 31/437; A61K 31/675; A61P 31/14

(52) U.S. Cl. CPC A61K 31/437; A61K 31/675; A61P 31/14

(57) ABSTRACT

One aspect described herein includes a method for treating coronavirus 2019 disease (COVID-19) caused by severe respiratory syndrome coronavirus 2 (SARS-CoV-2) by administering an effective amount of a DHODH inhibitor. Another aspect described herein includes a method of treating COVD-19 by administering 4-chlorophenyl (S)-6-chloro-1-(4-methoxyphenyl)-1,3,4,9-tetrahydro-2H-pyrido[3,4-b]indole-2-carboxylate (PTC299), or a pharmaceutically acceptable salt thereof.

### FIELD

The present description relates generally to active pharmaceutical ingredients for the treatment of subjects infected with RNA viruses, particularly viruses from the family Coronoviridae, more particularly SARS-CoV2 viruses. The present description also relates generally to the use of inhibitors of dihydroorotate dehydrogenase (DHODH) in the treatment of subjects infected with RNA viruses.

### BACKGROUND

### 1. COVID-19

Since the first patient with a pneumonia of unknown origin in late December 2019 in Wuhan City, China, a new coronavirus, designated SARS-CoV-2, has resulted in a rapidly spreading world-wide pandemic of respiratory illness termed coronavirus disease 2019 (COVID-19). SARS-CoV-2 is a positive sense RNA virus of zoonotic origin and is part of the coronavirus family. Due to its novelty and the apparent lack of population or group immunity, COVID-19 has been spreading rapidly in many countries across the globe causing great harm to human health, economic activity, and disrupting the social fabric on many levels (Wang 2020a). As of 08 Apr. 2020, 1,453,247 cases of COVID-19 and 83,588 deaths have been reported world-wide, with 402,47 cases and 12,914 deaths in the United States (Coronavirus Outbreak).

COVID-19 begins with SARS-CoV2 infection of the upper respiratory tract. Upon infection, the virus replicates rapidly, and after several days the infection extends to the lungs where the pathology becomes manifold and includes pneumonia, immune hyper-reactivity, pulmonary infiltration, and fibrosis leading to permanent lung damage and in some cases death. COVID-19 is considered to have two critical elements:
Uncontrolled SARS-CoV-2 replication, and
Overreactive immune-inflammatory response.

All ages are susceptible to infection with SARS-CoV-2 (Cheng 2020, Li 2020). The clinical features of COVID-19 are varied, ranging from an asymptomatic state to acute respiratory distress syndrome (ARDS), multi-organ dysfunction syndrome, and death (Chen 2020a, Huang 2020, Liu 2020a, Wang 2020b). According to current analyses, about 80% of the cases resolve with at-home care and supporting therapy and 20% require hospitalization of which 25% to 30% develop severe respiratory complications. The overall fatality from COVID-19 is estimated to range from 1% to 5% with a higher risk of death in the elderly and persons with underlying co-morbidities (50% to 70% of fatal cases) (Coronavirus Outbreak , CEBM 2020)

COVID-19 can be broadly divided into 3 stages (Shi 2020):
An asymptomatic incubation period;
Non-severe period of early stage typified by rapid viral proliferation; and
Severe respiratory symptomatic stage of the disease typified by a rise in inflammatory cytokines and the influence of comorbidities on disease progression and prognosis.

The common clinical features of COVID-19 include fever, cough, sore throat, headache, fatigue, myalgia, and shortness of breath. In a subset of patients, by the end of the first week, the disease can progress to pneumonia, respiratory failure, organ failure, and death. SARS-CoV-2 targets ciliated cells which are subsequently shed. The disease progression is also associated with an extreme rise in the level of inflammatory cytokines including interleukin (IL)-2, IL-7, IL-10, granulocyte-colony stimulating factor (GCSF), interferon gamma-induced protein 10 (IP-10), monocyte chemoattractant protein (MCP) 1, macrophage inflammatory protein (MIP)1A, and tumor necrosis factor alpha (TNFa), and vascular endothelial growth factor (VEGF) (Chen 2020a, Huang 2020). Both IL-6 and IL-17 have been implicated in acute respiratory distress syndrome (ARDS) (Wang 2020b, Xu 2020). Levels of IL-6 appear to play an important role in COVID-19 disease severity as there is strong evidence that IL-6 peak levels are associated with severity of pulmonary complications (Russell 2020).

The hyper induction of pro-inflammatory cytokines - also referred to as a “cytokine storm” or cytokine release syndrome (CRS) - is common in acute viral infections and can lead to tissue damage and pulmonary pathology (Wong 2004, Zhang 2004, Yuen 2005, Chien 2006). VEGF, which plays a role in the pathogenesis of ARDs, has also been found to be increased in patients with COVID-19 (Liu 2020b). The viral load of SARS-CoV2 detected in a patient’s respiratory tract correlates with lung disease severity (Liu 2020b).

To date, there are no specific therapeutic agents for COVID-19 and currently treatment is supportive and based on symptomatology (Chen 2020b, Jin 2020). A therapeutic agent that targets both viral replication and the hyper-reactive immune response would offer a highly desirable treatment for COVID-19 management.

### 2. DHODH Inhibitors and Antiviral Activity

All viruses are dependent upon their host for cellular machinery and substrates required for viral proliferation. RNA viruses, including SARS-CoV-2, require a large intracellular nucleotide pool to support rapid viral RNA replication and proliferation. Coronaviruses have the largest viral genomes known to date (ranging from 26 to 32 kB); SARS-CoV-2 has a genome of ~30 kB) (Wu 2020). Thus, a reduction in the availability of nucleotides for de novo viral synthesis may inhibit the replication of SARS-CoV-2 and potentially limit viral burden in the patient.

Dihydroorotate dehydrogenase (DHODH) is located on the inner membrane of mitochondria and acts in the de novo pyrimidine nucleotide synthesis pathway to catalyze dehydrogenation of dihydroorotate (DHO) to orotic acid (ORO), resulting in the generation of uridine monophosphate (UMP) (Munier-Lehmann 2013). UMP is subsequently converted to uridine (U) and cytosine (C) triphosphates to supply the cellular pool of pyrimidine nucleotides.

Under normal conditions, pyrimidine nucleotides are supplied via both de novo biosynthesis and salvage pathways, as illustrated in FIG. 1. The salvage pathway recycles nucleotides from food or other extracellular sources and does not appear to be sufficient to support viral proliferation.

Several compounds that inhibit DHODH enzyme activity have been shown to have broad-spectrum antiviral activity (Lucas-Hourani 2015, Munier-Lehmann 2015, Lewis 2016, Diedrichs-Mohring 2018, Lolli 2018, Sykes 2018, Chen 2019, Mei-jiao 2019, Xiong 2020). This is due to the central role of DHODH in pyrimidine nucleotide synthesis. DHODH inhibitors are particularly active against RNA viruses of both positive and negative strand genome polarity and have shown efficacy against coronaviruses and a number of other respiratory RNA viruses. See Table 1 below for a list of Human RNA viruses whose replication is reported to be inhibited by DNODH inhibitors.

TABLE 1

| | | | | | |
|---|---|---|---|---|---|
| Positive sense RNA viruses | References | Negative sense RNA viruses | References | Double stranded RNA viruses | References |
| SARS-CoV-2 | (Cheung 2017, Xiong 2020) | Influenza virus A/B | (Hoffmann 2011, Cheung 2017, Xiong 2020) | Rotavirus | (Chen 2019) |
| SARS-CoV | (Cheung 2017) | Respiratory syncytial virus | (Dunn 2011) | | |
| MERS-CoV | (Cheung 2017) | Measles virus | (Lucas-Hourani 2013, Lucas-Hourani 2017) | | |
| Human coronavirus 229E | (Lucas-Hourani 2017) | Ebola virus | (Luthra 2018) | | |
| Rhinovirus | (Cheung 2017) | Arenaviruses | (Ortiz-Riano 2014) | | |
| Zika virus | (Luthra 2018, Xiong 2020) | | | | |
| Chikungunya virus | (Lucas-Hourani 2013) | | | | |
| Coxsackievirus B3 | (Lucas-Hourani 2013) | | | | |
| West Nile virus | (Hoffmann 2011, Lucas-Hourani 2013) | | | | |
| Dengue virus | (Hoffmann 2011, Wang 2011, Yang 2018) | | | | |
| Hepatitis C virus | (Hoffmann 2011) | | | | |

A recent study by Xiong et al (2020), one of the studies cited in Table 1, evaluated the anti-viral activity of 2 new inhibitors of DHODH, S312 and S416, and the previously known DHODH inhibitor, teriflunomide. These inhibitors were effective against multiple RNA viruses in vitro and particularly against SARS-CoV-2 (Xiong 2020). The half maximal effective concentration (EC₅₀) for SARS-CoV-2 ranged from 0.017 to 26.1 µM. The authors hypothesized that the high potency observed was due to the makeup of the SARs-CoV-2 genome; SARS-CoV-2 contains approximately 32% of uridine in its genome explaining why DHODH inhibitors are particularly effective in stopping SARS-CoV-2 replication.

Most existing direct-acting antiviral drugs cannot be applied immediately to new viruses because of virus-specificity of their action and also because development of these types of drugs from the beginning is often ill-timed for outbreaks (Xiong 2020). Nevertheless, DHODH inhibitors may be particularly effective because the accumulation of viral mutations is not likely to alter the fundamental viral need for UTP.

### 3. DHODH Inhibitors and Cytokine Storms

As described in section 1 (COVID-19) above, acute viral infections can cause severe complications associated with hyper induction of pro-inflammatory cytokines (Yokota 2003, Yuen 2005). This inflammation can contribute to significant tissue damage, lung disease, multi-organ failure and mortality (Yoshikawa 2009). That type of excessive immune response common to such viral infections due to increased cytokine expression is referred to as a “cytokine storm.” This hyperactive immune response results in an extreme inflammatory response, causing pulmonary and multi-organ dysfunction, ultimately leading to multiple organ failure. In the lung, in particular, the inflammatory response can lead to fatal pneumonia and pulmonary infiltration and downstream fibrosis which can require ventilatory support. Lung failure due to inflammation and immune system-mediated damage is the leading cause of mortality in SARS-CoV-2 infection. Since DHODH inhibitors target a host protein, they can be expected to retain antiviral activity against all strains of SARS-CoV-2 including variants.

Data suggest DHODH inhibitors reduce cytokine storms (Xiong 2020). In a mouse model of influenza virus infection, mice treated with a DHODH inhibitor had significantly reduced levels of proinflammatory cytokines including interferon-gamma, MCP-1, IL-5, and IL-6 (Xiong 2020). The DHODH inhibitor leflunomide reduces the release of pro-inflammatory cytokines (Li 2013) and suppresses TNF-induced cellular responses (Breedveld 2000).

DHODH inhibitors could play a similar immune-regulating role in COVID-19 patients (Xiong 2020).

In other words, COVID19 can broadly be described by 3 stages (Shi 2020):
An asymptomatic incubation period;
An early stage, non-severe period typified by rapid viral proliferation; and
A severe respiratory symptomatic stage of the disease typified by a rise in inflammatory cytokines and the influence of comorbidities on disease progression and prognosis.

A large pool of nucleotides is essential to support rapid SARS-CoV-2 proliferation during the early stages of COVID-19 infection. Inhibiting DHODH activity could starve the virus of pyrimidine nucleotides essential for viral RNA replication. Consistent with this hypothesis, multiple DHODH inhibitors are known to be potent broad-spectrum antiviral compounds and to be active against SARS-CoV-2.

DHODH inhibitors are also known to block cytokine storms and likely may act to reduce this hyper-immune response in patients with COVID-19.

In summary, inhibiting DHODH activity could negatively impact both stages of a COVID-19 infection by:
Blocking acute SARS-CoV2 viral replication; and
Suppressing the chronic inflammatory phase due to a stress-induced overreactive immune response

There is an immense and unmet need for effective treatments against SARS-CoV-2 viral infections. What is needed is a drug or combination of drugs with a good safety and efficacy profile that can inhibit replication of the virus and/or control an overactive immune response. It is thought that some DHODH inhibitors may well be efficacious, for reasons provided above, however, to date no such compound has been approved for treatment of such infections.

### SUMMARY

In one aspect described herein, there is provided a method for treating coronavirus 2019 disease (COVID-19) caused by severe respiratory syndrome corona virus 2 (SARS-CoV-2) by administering an effective amount of 4-chlorophenyl (S)-6-chloro-1-(4-methoxyphenyl)-1,3,4,9-tetrahydro-2H-pyrido[3,4-b]indole-2-carboxylate, a pharmacologically active enantiomer (hereinafter referred to as “PTC299”), having the structure:

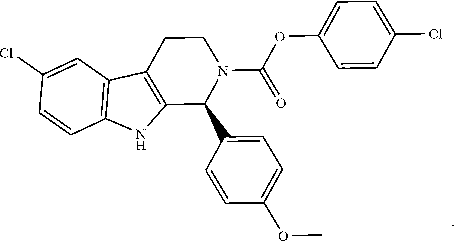

or a pharmaceutically acceptable salt thereof to a patient in need thereof.

In one aspect, administration of the effective amount of PTC299 results in inhibition of cytokine storms in the patient.

In another aspect, administration of the effective amount of PTC299 results in inhibition of replication of SARS-CoV-2.

In another aspect described herein, there is provided a method for ameliorating coronavirus 2019 disease (COVID-19) caused by severe respiratory syndrome corona virus 2 (SARS-CoV-2) by administering an effective amount of PTC299 or a pharmaceutically acceptable salt thereof to a patient in need thereof.

In another aspect described herein, there is provided a method for controlling the transmission of coronavirus 2019 disease (COVID-19) caused by severe respiratory syndrome corona virus 2 (SARS-CoV-2) by administering an effective amount of PTC299 or a pharmaceutically acceptable salt thereof to a patient in need thereof.

In another aspect described herein, there is provided a method for inhibiting viral replication of SARS-CoV-2 by administering an effective amount of PTC299 or a pharmaceutically acceptable salt thereof to a patient in need thereof.

In another aspect described herein, there is provided a method for reducing replication or proliferation of SARS-CoV-2 by administering an effective amount of PTC299 or a pharmaceutically acceptable salt thereof to a patient in need thereof.

In another aspect described herein, there is provided a method of inhibiting cytokine storms in a subject infected with SARS-CoV-2 comprising administering an effective amount of PTC299 or a pharmaceutically acceptable salt thereof to the subject.

It will be understood that in any or all of the above aspects remdisivir can be administered in a therapeutically effective amount in combination with the 4-chlorophenyl (S)-6-chloro-1-(4-methoxyphenyl)-1,3,4,9-tetrahydro-2H-pyrido[3,4-b]indole-2-carboxylate.

In another aspect of the method, PTC299 is administered in combination with at least one other antiviral agent. In another aspect, the at least one other antiviral agent is remdesivir.

Another aspect described herein is a method of inhibiting or reducing replication of SARS-CoV-2 in a mammalian cell infected therewith by contacting the cell with an effective amount of PTC299 or a pharmaceutically acceptable salt thereof.

### BRIEF DESCRIPTION OF THE DRAWINGS

FIG. 1 is a diagram showing two pathways for pyrimidine nucleotide synthesis, Salvage Pathway and a De novo Pathway. Abbreviations in the figure are as follows: CAD, complex of the following three enzymes: carbamoyl-phosphate synthetase 2, aspartate carbamoyltransferase, and dihydroorotase; CDA, cytidine deaminase; CMP, cytosine monophosphate; CTP, cytosine triphosphate; DHO, dihydroorotate; UDP, uridine diphosphate; UMP, uridine monophosphate; UPP, uridine phosphorylase; UTP, uridine triphosphate; UMPS, uridine monophosphate synthase; and dCTP, deoxycytidine triphosphate.

FIG. 2 shows the chemical structure of two enantiomers, an active S-enantiomer (PTC299) and an inactive R-enantiomer (PTC-371).

FIG. 3 is a plot of % inhibition vs compound concentration in nM, illustrating the results of a colorimetric assay of inhibition of DHODH activity in vitro in mitochondria extracts by PTC299, brequinar, and teriflunomide, compared to the negative control, PTC-371.

FIG. 4 is a bar graph showing the percent inhibition of de novo pyrimidine synthesis of ¹⁵N-CTP, ¹⁵N-UTP, and ¹⁵N-UMP in HT 1080 cells by PTC299 compared to the corresponding inactive enantiomer, PTC-371.

FIG. 5 is a plot showing the fraction of total blasts relative to DMSO control vs PTC299 concentration in nM, from an assay of the ability of PTC299 to suppress DHODH activity in the presence of increasing concentrations of uridine (0, 3, 10, 30, or 100 µM) .

FIG. 6 consists of two bar graphs illustrating the results of assays of the inhibition of DHODH activity in patients with neurofibromatosis Type 2 tumors. FIG. 6A illustrates the serum dihydroorotate concentration from a total of 5 patients pre- and post-dosing with PTC299 for up to 16 weeks. FIG. 6B illustrates the mean serum VEGF plasma levels from 11 patients pre- and post- PTC299 dosing for 8 weeks.

FIG. 7 consists of a plot of log ratio results from a study of the effect of four concentrations of PTC299 (100, 10, 1 and 0.1 nM) on cytokine production using BioMAP profiling (BioSeek, now part of Eurofins), consisting of evaluation in four models of primary cell disease). The x-axis indicates the cytokines evaluated and the y-axis indicates the log ratio of PTC299 treated vs vehicle control. FIG. 7A shows cytokine production profile results from peripheral blood mononuclear cells (PBMCs) plus venular endothelial cells (sAg). FIG. 7B shows results from PBMCs plus B cells (BT:B Cell). FIG. 7C illustrates results from PBMCs plus fibroblasts (HDFSAg). FIG. 7D illustrates results from T_{H2} cells plus venular endothelial cells (/TH2).

FIG. 8 is a bar graph of % inhibition of cytokine release from six different human Th cytokines (IL-10, IL-17A, IL-17F, IL-5, IL-6 and IL-9) relative to DMSO measured using a LEGEND-lex® Assay Kit (FACS-based) in the presence of three different concentrations of PTC299 (1, 10 and 100 nM).

FIG. 9 depicts two plots of % inhibition vs. compound concentration of results of a study of inhibition of IL17 and IL17 F production by increasing concentrations of PTC299 compared to PTC-371, A77 and brequinar. FIG. 9A is a plot of results of IL17 production in PBMC with cytokine stimulation over 88 hours. FIG. 9B is a plot of results of IL17 F production in PBMC with cytokine stimulation over 88 hours.

FIG. 10 is a plot of PTC299 plasma concentration (µg/mL) vs hours of plasma dihydroorotate (DHO) and PTC299 concentrations after a single dose of PTC299 was administered to a single monkey.

FIG. 11 depicts two plots of results of simulated desmethyl PTC299 maximum concentration (Cₘₐₓ) (FIG. 11A) and O-desmethyl PTC299 area under the concentration curve from 0 to 24 hours (AUC₀₋₂₄) (FIG. 11B) vs maximum ALT in selected individuals with varying hepatotoxicity sensitivity at day 112 and O-desmethyl PTC299 AUC₀₋₂₄ maximum ALT in three selected individuals with varying hepatotoxicity sensitivity at day 112 from Sim Cohorts v4A-1-Multi16_ID_2 (diamond symbols, the most sensitive individual), Multi16 ID_10 (square symbols, the individual with intermediate sensitivity), and Multi16_ID_15 (triangle symbols, the least sensitive individual). Light and dark shades represent maximum ALT > 1X and 3X ULN, respectively. Black arrows represent the minimum desmethyl PTC299 Cₘₐₓ or AUC₀₋₂₄ₕᵣ that showed max ALT > 1X or 3X ULN among the simulated individuals.

FIG. 12 is a set of four plots of results of viral titer vs time results of four plates of Vero cells pretreated with one of four concentrations of PTC299 (0.25, 0.5, 1, or 0 µM) alone or with uridine for different pretreatment times, as described in Example 8. FIG. 12A is a plot of results from 18 hours of pretreatment with PTC299. FIG. 12B is a plot of results from 18 hours of pretreatment with PTC299 and uridine. FIG. 12C is a plot of results from 2 hours of pretreatment with PTC299. FIG. 12D is a plot of results from 2 hours of pretreatment with PTC299 and uridine.

FIG. 13 is a set of images of DAPI stained Vero cells, where viral levels were determined by staining cells using the N-protein antibody, as reflected by green fluorescence. FIG. 13A shows cells that were uninfected. FIG. 13B shows cells infected with SARS-CoV-2 and treated with DMSO. FIG. 13C shows cells infected with SARS-CoV-2 and treated with 30 nm PTC299. FIG. 13D shows cells infected with SARS-CoV-2 and treated with 300 nm PTC299. FIG. 13E shows cells infected with SARS-CoV-2 and treated with 3 µM PTC299.

FIG. 14 is a set of two plots of SARS-CoV-2 (SCoV2) titer in Vero cells via 50% tissue culture infections dose assay (TCID₅₀/mL) vs hours post infection after treatment with 1 (diamond), 0.5 (square), 0.25 µM of PTC299 or untreated (x). FIG. 14A is a plot of results obtained with 2 hours of pre-treatment of PTC299. FIG. 14B is a plot of results obtained with 18 hours of pre-treatment with PTC299.

FIG. 15 is a plot of SARS-CoV-2 titer (log TCID₅₀/mL) vs PTC299 concentration (log nM), from a dose response assay used to determine 50% and 90% effective concentrations (EC₅₀ and EC₉₀, respectively).

FIG. 16 is a plot of percent cytotoxicity (ATP) vs concentration of PTC299 from an assay of the effect of PTC299 on Vero cells.

FIG. 17 is a plot of SARS-CoV-2 titer (log TCID₅₀/mL) vs hours post infection (h.p.i.) of infected Vero Cells either untreated (circle) or treated with 5 µM remdesevir (square), 2.5 µM remdesevir (triangle), 500 nM PTC299 (inverted triangle) or 250 nM PTC299 (diamond). The results show that PTC299 and remdesevir each reduce the replication of SARS-CoV-1 in Vero Cells.

FIG. 18 is a plot of SARS-CoV-2 titer (log TCID₅₀/mL) vs hours post infection (h.p.i.) of infected Vero Cells either untreated (circle) or treated with 5 µM remdesevir (light square), 500 nM PTC299 (inverted triangle), 250 nM PTC299 (diamond), 2.5 µM remdesevir (dark square) plus 500 nM PTC299 or 2.5 µM remdesevir (dark square) plus 250 nM PTC299 (triangle). The results show that remdesivir (2.5 µM) enhances the antiviral activity of PTC299.

FIG. 19 is a plot of SARS-CoV-2 titer (logTCID₅₀/mL) vs hours post infection (h.p.i.) of infected Vero Cells either untreated (circle) or treated with 5 µM remdesevir (first square), 500 nM PTC299 (inverted triangle), 250 nM PTC299 (diamond), 5 µM remdesevir plus 500 nM PTC299 (triangle) or 5 µM remdesevir plus 250 nM PTC299 (second square). The results show that PTC299 in combination with remdesivir (5 µM) potently inhibits SARS-CoV-2 replication in Vero Cells.

### DETAILED DESCRIPTION

### 1. Chemical Structure and Background of PTC299

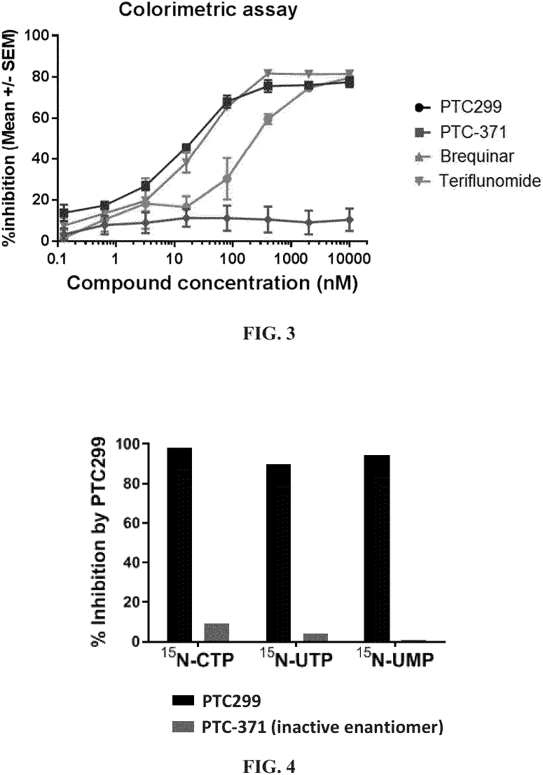

4-chlorophenyl-(S)-6-chloro-1-(4-methoxyphenyl)-1,3,4,9-tetrahydro-2H-pyrido[3,4-b]indole-2-carboxylate (PTC299), and a suitable method of making the same were disclosed in the international patent application published as WO2005/089764. PTC299 is a 6-chloro-β-carboline derivative and has a chemical formula of C₂₅H₂₀C₁₂N₂O₃ and a molecular weight of -467 Daltons. PTC299 is the S-enantiomer. The R-enantiomer is inactive. The structure of the two enantiomers is shown in FIG. 2. The inactive R-enantiomer is also referred to herein below as PTC-371.

PTC299 was initially identified as a member of a genus of compounds that act as inhibitors of human vascular endothelial growth factor (VEGF) production, that can be useful in the inhibition of angiogenesis, and/or in the treatment of cancer, diabetic retinopathy or exudative macular degeneration (WO2005/089765). That application discloses a method for making the compound. International patent application published as WO2010/138758 disclosed PTC299, and other compounds, could be used in methods for treating cancer and non-neoplastic conditions, as well as inhibiting viral replication, viral RNA, DNA or protein production. That application provides examples on the use of the compound in the inhibition of a broad range of viruses, including vaccinia, adenovirus, herpes simplex virus-1 (HSV-1), Influenza A, Parainfluenza, respiratory syncytial virus (RSV), Yellow Fever, Dengue 2, West Nile Virus (WNV), and polio virus (PV). Use of PTC299, and other compounds, for selectively inhibiting viral replication is also illustrated in the international patent application published as WO2011/150162. More recently, PTC299 has been determined to be a potent inhibitor of dihydroorotate dehydrogenase (DHODH), see, for example, international patent application having the publication number WO2019028171, in which PTC299 is identified as being useful in the treatment of hematologic cancers, such as leukemia or myelodysplastic syndrome.

As an inhibitor of DHODH, PTC299 is provided for the treatment or amelioration of COVID-19 at multiple stages.

PTC299 may act through a dual mechanism:
Inhibiting viral proliferation
Blocking cytokine release syndrome triggered by the virus

Without wishing to be bound by any theory, PTC299 is useful for the treatment or amelioration of COVID-19 for at least the following reasons:
PTC299 is an inhibitor of cellular DHODH enzyme activity.
   DHODH inhibitors have been shown to inhibit SARS-CoV-2 replication.
PTC299 has demonstrated inhibition of
   PTC299 inhibits a host cellular protein, and similar to other host targeting antivirals, has broad antiviral activity, thereby increasing the likelihood it will be effective against all current and future strains of SARS-CoV-2.
   Host targeting antivirals have advantages over direct virus-acting antivirals as they are not virus-specific - hence they are effective even if the virus mutates, thus minimizing the emergence of viral resistance.

PTC299 also attenuates excessive cytokine release. In addition to the above, PTC299 has been shown in previous studies to be generally well tolerated over a range of dosing regimens. PTC299 was extensively evaluated in 9 clinical studies (n=169 in healthy volunteers and n=134 in solid tumor cancer patients) and is currently being evaluated in patients with acute myeloid leukemia (AML); the AML study has enrolled 14 patients to date. Also, data in patients treated with PTC299 have demonstrated increases in serum dihydroorotate (DHO) following treatment, indicating that in vivo PTC299 demonstrates target engagement and elicits appropriate pharmacodynamic changes.

The key characteristics set forth above indicate that PTC299 can address two critical aspects of COVID-19 pathology - viral replication and immune-inflammatory responses, particularly in the lung.

### 2. Combinations

In another aspect, PTC299 is used in combination with at least one other antiviral agent to treat COVID-19 caused by SARS-CoV-2 or to inhibit replication of SARS-CoV-2 in a subject by administering a therapeutic amount of the combination to a subject in need thereof. In one aspect the at least one other antiviral agent is selected from the group consisting of acyclovir, adefoir, amantadine, ampligen, amprevanir, umifenovir, atazanavir, atripla, baloxavir marboxil, biktarvy, boceprevir, bulevirtide, cidofovir, cobicistat, combivir, caclastavir, darunavir, delavirdine, descovoy, didanosine, docosanol, delaviridine, descovoy, didanosine, docosanol, dolutegravir, doravirine, edoxudine, efarirenz, elvitegravir, emtricitabine, enfuvirtide, entecavir, etravirine, famcilovir, fomivirsen, fosambrenavir, foscarnet, ganciclovir, ibacitabine, ibalizumab, idoxuridine, imiquimod, imuvonir, indinavir, lamivudine, letermovir, lopinavir, loviride, maraviroc, methisazone, moroxydine, nelfinavir, maraviroc, methisazone, moroxydine, nelfinavir, nefvirapine, nexavir, nitazozanide, norvir, oseltamivir, penciclovir, peramivir, penciclovir, peramivir, pleconaril, podophyllotoxin, raltegravir, remdesivir, ribavirin, rilpiririne, pipivirine, rimantadine, ritonavir, saquinavir, simeprevir, sofosfuvir, stavudine, taribavirin, telaprevir, telbivudine, tenfovir alafenamide, tenofir disoproxil, tipranavir, trifluridine, trizivir, tromantadine, Truvada, umifenovir, valaciclovir, valganciclovir, vicriviroc, vidarbanine, zalcitabine, zanamivir, and zidovudine. In another aspect the at least one other antiviral agent is remdesivir or ribovarin.

In another aspect, PTC299 is used in combination with remdesivir to treat COVID-19 caused by SARS-CoV-2 or to inhibit replication of SARS-CoV-2 in a subject by administering the combination to a subject in need thereof.

### 3. PTC299 Mechanism of Action

PTC299 is an orally bioavailable small molecule originally developed as an agent for the treatment of solid tumors. The mechanism of action of PTC299 is due to its direct and potent inhibition of DHODH.

Similar to other DHODH inhibitors, PTC299 demonstrates broad-spectrum antiviral activity against RNA viruses in vitro, as illustrated in international patent application publication number WO2010/138758, and as illustrated in other studies cited in Table 2, below:

TABLE 2

| | |
|---|---|
| Positive sense RNA viruses EC₅₀ (nM) | Negative sense RNA viruses EC₅₀ (nM) |
| West Nile virus (Vero cells)^{b} 10 | Respiratory syncytial virus 4 (Hep2 cells)^{b} |
| Hepatitis C virus (replicon, 36 Huh7 cells)^{C} | Ebola virus (Vero cells)^{C} 9 |
| Poliovirus (HeLa cells)^{a} 0.6 | Rift Valley fever virus 13 (HeLa cells)^{C} |

Source: ^{a}Data on file (PTC Therapeutics, Inc.); ^{b}SRI Study #12852.02; “Data on file, studies done at USAMRID.

DHODH inhibitors have been demonstrated to act as broad-spectrum antivirals having the ability to inhibit viral replication and survival of a range of both RNA and DNA viruses. Inhibition of DHODH results in the depletion of the intra-cellular pyrimidine nucleotide pool necessary for viral proliferation. DHODH inhibitors also inhibit cytokine storms.

Without wishing to be bound by any particular theory, PTC299 is useful for treating or ameliorating COVID-19 for at least the following reasons:
PTC299 is an inhibitor of cellular DHODH enzyme activity.
   DHODH inhibitors have been shown to inhibit SARS-CoV2.
PTC299 has demonstrated inhibition of SARS-CoV-2 replication in vitro:
   PTC299 inhibits a cellular host factor and, similar to other host targeting antivirals, has broad-spectrum antiviral activity, increasing the likelihood it will be effective against all current and future strains of SARS-CoV-2.
   Host targeting antivirals have advantages over direct virus-acting antivirals as they are not virus-specific - hence they are effective even if the virus mutates, thus minimizing the emergence of viral resistance.
PTC299 inhibits cytokine storms due to viral infection that can cause tissue damage, pneumonia, and organ failure.
   PTC299 reduces levels of IL-6 along with other cytokines; elevated levels of IL-6 are positively associated with severity of pulmonary complications.
PTC299 has been extensively evaluated in clinical studies (n=168 in healthy volunteers and n=134 in cancer patients) and has been shown to be generally well-tolerated over a range of dosing regimens and at doses higher than proposed for COVID-19 treatment.

The COVID-19 pandemic is a large unmet medical need. The mechanism of action of PTC299 and the fact it has broad-spectrum antiviral activity and is likely to inhibit SARS-CoV-2, b) reduces cytokine storms, and c) is well-tolerated indicates there is a positive benefit/risk profile for evaluating the drug in patients with COVID-19.

### 4. Dosage Form

In one aspect, PTC299 is provided in any suitable and effective formulation type, dosage form, and for any suitable route of administration. In another aspect, PTC299 is provided in an oral dosage form selected from a tablet, a suspension, a powder, or a capsule.

In another aspect, PTC299 is provided in a bioavailable dosage form comprising a spray dried intermediate, such as a dosage form disclosed in an international patent application published as WO2020028778. In that aspect, the spray dried intermediate is suitably present in the form of a liquid capsule or an oral tablet comprising PTC299 and at least one pharmaceutically acceptable excipient. In another aspect, the PTC299 is provided in the form of a tablet.

Any suitable amount of PTC299 can be included in a dosage form administered according to a method of the invention. A suitable amount is a therapeutically effective amount. As used herein, therapeutically effective amount is an amount effective to treat or ameliorate COVID-19 caused by SARS-CoV-2 in a patient or to inhibit viral replication of SARS-CoV-2 or an amount sufficient to reduce replication or proliferation of SARS-CoV-2 or to inhibit cytokine storms in a patient. For example, a suitable amount can be an amount sufficient to reduce replication or proliferation of SARS-CoV-2 by a percentage amount such as, for example, by 10% , 20%, 30%, 40%, 50%, 60%, 70%, 80%, 90% or more.

Doses administered on a weight basis may be in the range of about 0.001 mg/kg/day to about 3500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 600 mg/kg/day, or about 0.01 mg/kg/day to about 500 mg/kg/day, or about 0.01 mg/kg/day to about 300 mg/kg/day, or about 0.015 mg/kg/day to about 200 mg/kg/day, or about 0.02 mg/kg/day to about 100 mg/kg/day, or about 0.025 mg/kg/day to about 100 mg/kg/day, or about 0.03 mg/kg/day to about 100 mg/kg/day, wherein said amount is orally administered once (once in approximately a 24 hour period), twice (once in approximately a 12 hour period) or thrice (once in approximately an 8 hour period) daily according to subject weight. In certain embodiments, the effective amount will be in a range of from about 0.001 mg/kg/day to about 500 mg/kg/day, or about 0.01 mg/kg/day to about 500 mg/kg/day, or about 0.1 mg to about 500 mg/kg/day, or about 1.0 mg/day to about 500 mg/kg/day, in single, divided, or a continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg.

PTC 299 can be included in a dosage form in an amount, for example, from about 5 mg per unit dosage form to about 200 mg per unit dosage form. Examples of suitable amounts of PTC299 included in such a dosage form include, but are not limited to 200 mg, 100 mg, 50 mg, 10 mg. or 5 mg per unit dosage form. PTC 299 can be provided in a dosage form in an amount from about 0.25 µm to about 1.0 µm.

When PTC299 is administered in combination with another antiviral agent, the amount of PTC299 and other antiviral agent are such that the combination is effective to treat or ameliorate COVID-19 caused by SARS-CoV-2 in a patient or to control transmission of COVID-19 caused by SARS-CoV-2 or to inhibit viral replication of SARS-CoV-2 or an amount sufficient to reduce replication or proliferation of SARS-CoV-2 or an amount sufficient to inhibit a cytokine storm. For example, a suitable amount can be an amount sufficient to reduce replication or proliferation of SARS-CoV-2 by a percentage amount such as, for example, by 10%, 20%, 30%, 40%, 50%, 60%, 70%, 80%, 90% or more.

When remdesivir is used in combination with PTC299, remdesivir can be provided at a concentration of, for example, from about 2.5 µM to about 5 µM and PTC299 can be provided at a concentration of from about 250 nM to about 500 nM.

When remdesivir is used in combination with PTC299, the molar ratio of remdesivir to PTC 299 can be any suitable molar ratio. For example, the molar ratio of remdesivir to PTC299 can be about about 50:1 to about 2:1, about 40:1 to about 2:1, about 30:1 to about 3:1, about 20:1 to about 3:1, about 15:1 to about 5:1, about 5:1, about 20:1, about 10:1 or about 5:1.

The present invention is described in more detail with reference to the following nonlimiting examples, which are offered to more fully illustrate the invention, but are not to be construed as limiting the scope thereof. The examples illustrate the usefulness of PTC299 in the inhibition of DHODH, in reducing SARS-CoV-2 replication, and/or in controlling the inflammatory response.

### EXAMPLES

### Example 1 - Colorometric Assay Demonstrating PTC299 Is a DHODH Inhibitor

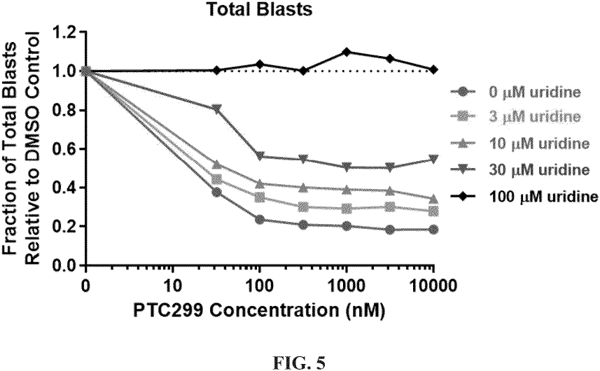

In vitro studies have demonstrated that PTC299 inhibits de novo pyrimidine nucleotide biosynthesis and selectively binds DHODH from mitochondrial extracts. These studies also showed PTC299 inhibits purified recombinant DHODH activity present in isolated mitochondria. In this example, an isolated mitochondria colorimetric assay was conducted comparing the percent inhibition resulting from administration of 0.1, 1, 10, 100, 1000, or 1000 nM of PTC299, PTC-371 (inactive enantiomer), brequinar, and teriflunomide. The latter two compounds are known DHODH inhibitors. The inhibition was specific to PTC299 as no inhibition was observed using the inactive R-enantiomer PTC371. The inhibitory effect of PTC299 on DHODH was similar to or greater than that of the known DHODH inhibitors brequinar and teriflunomide (Chen 1992, Bar-Or 2014), as illustrated in the assay results shown in FIG. 3.

### Example 2 - Assay of Fibrosarcoma Cells Demonstrating PTC299 Is a DHODH Inhibitor

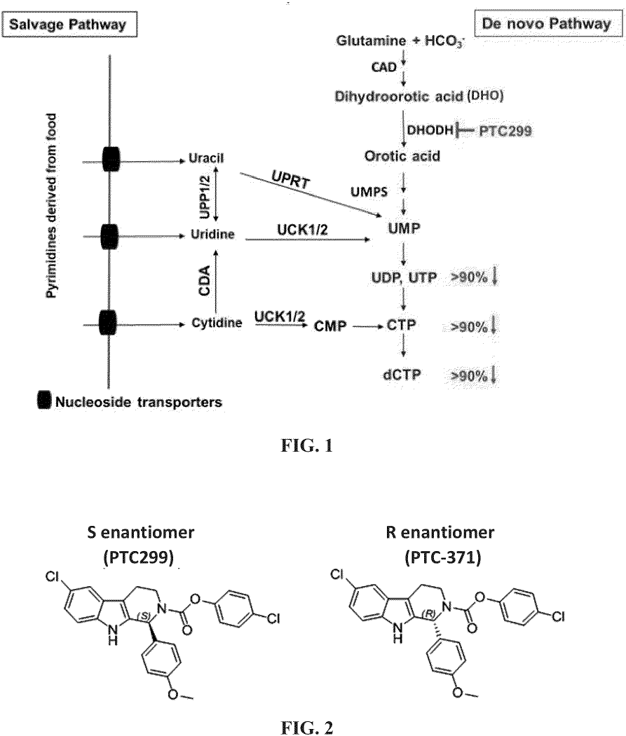

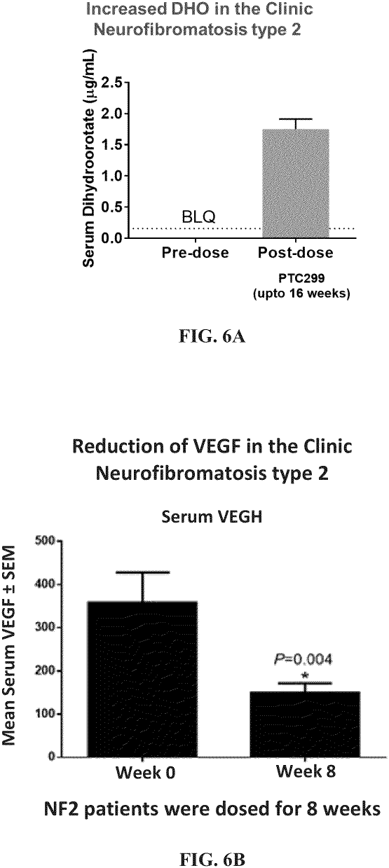

Cellular assays have also indicated that PTC299 inhibits de novo pyrimidine nucleotide synthesis through inhibiting DHODH activity. In this Example, fibrosarcoma cells were used to investigate the effect of PTC299 on pyrimidine nucleotide synthesis. Glutamine is an upstream precursor of dihydroorotic acid in the de novo pyrimidine synthesis pathway, as illustrated in FIG. 1. Addition of ¹⁵N-glutamine to HT1080 human fibrosarcoma cells resulted in the generation of pyrimidine nucleotides ¹⁵N-UTP and ¹⁵N-CTP, as illustrated in FIG. 4. As expected for a DHODH inhibitor, addition of PTC299 (100 nM) resulted in suppression of de novo pyrimidine nucleotide biosynthesis (FIG. 4). This inhibition is specific to PTC299 as treatment with inactive R-enantiomer of PTC-371 (100 nM) did not suppress de novo pyrimidine nucleotide biosynthesis (FIG. 4).

### Example 3 - Assay of AML Blast Cells Demonstrating PTC299 as a DHODH Inhibitor

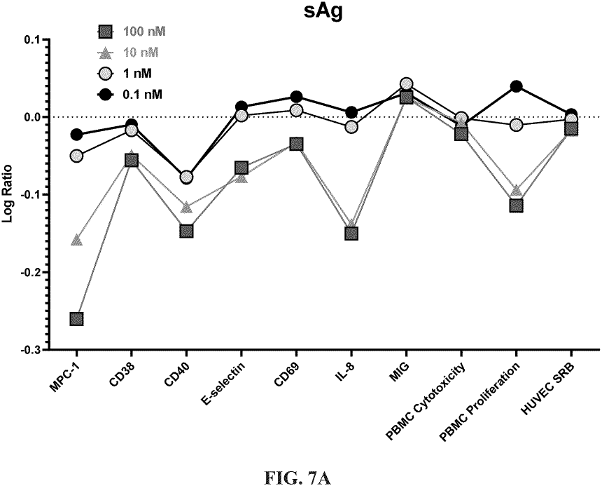

The ability of PTC299 to inhibit DHODH was further tested using primary acute myeloid leukemia (AML) blast cells that are reliant upon the de novo pyrimidine nucleotide pathway to obtain pyrimidine nucleotides. In these cells, PTC299 caused a G1/S cell-cycle arrest, inhibition of proliferation, and cell death (Cao 2020). The reversal of the effects of PTC299-dependent cytotoxicity by the addition of exogenous uridine indicate that this is a DHODH-dependent effect, as illustrated in FIG. 5.

In summary the evidence from Examples 1-3, above, demonstrate that PTC299 inhibits the activity of DHODH in each of the following contexts:
PTC299 inhibits DHODH in mitochondrial lysates
PTC299 inhibits the de novo conversion of ¹⁵N-glutamate to ¹⁵N-pyrimidine nucleotides
Uridine blocks the PTC299-mediated inhibition of DHODH activity

### Example 4 - PTC299 Activity in Oncology Patients

PTC299 has been extensively evaluated in 9 clinical studies; 5 were in oncology (n=134) and 4 were in healthy volunteers (n=169). Across these studies, PTC299 was shown to be generally well tolerated over a range of dosing regimens.

DHODH converts DHO to orotate (illustrated in the de novo pathway of FIG. 1). If PTC299 inhibits DHODH, it would be anticipated that DHO plasma levels would be increased in patients receiving the drug. In support of this, patients with a deficiency in DHODH enzyme activity have elevated serum DHO levels (Duley 2016).

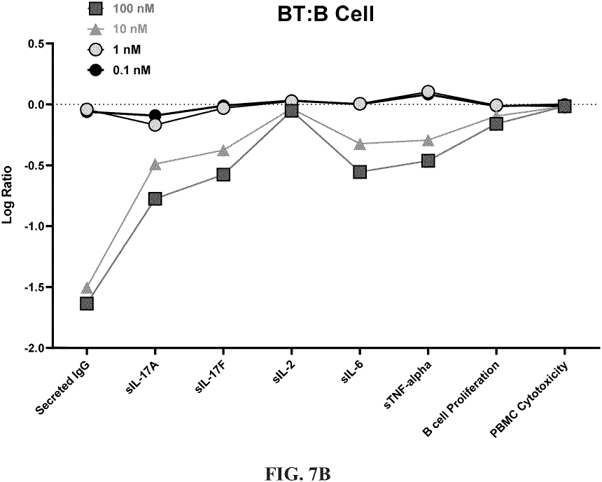

PTC299-ONC-007-NF2 (Study 007) was a Phase 2 study evaluating PTC299 monotherapy in adult patients with neurofibromatosis type 2. PTC299 was administered at a dose of 100 mg BID. Pre- and post-PTC299 treatment serum samples were available for 4 patients. Post-treatment samples were available after 8 weeks or after 16 weeks of treatment. As shown in FIG. 6A, serum DHO levels were below the lower limit of quantification prior to dosing and elevated at 8 or 16 weeks, with the differences between pre- and post-dosing DHO levels reaching statistical significance. The elevation of DHO levels was at least similar if not greater than the levels (0.537 ug/mL) observed in a patient with Miller syndrome who had a mutation in DHODH gene that results in loss of enzyme activity and disease phenotype (Duley 2016). These results were consistent with PTC299 strongly inhibiting DHODH activity in vivo.

The role of PTC299 as a DHODH inhibitor was additionally tested by examining if the drug could inhibit the production of VEGF. VEGF levels are known to be abnormally elevated in these cancer patients (Plotkin 2009). DHODH inhibitors have been shown to suppress VEGF expression (Diedrichs-Mohring 2018). As shown in FIG. 6B, serum VEGF levels were elevated prior to dosing but were significantly reduced after 8 weeks of treatment to levels similar to those reported for control subjects (Raimondo 2001, Kut 2007).

These data showing increases in serum DHO and decreases in serum VEGF indicate that PTC299 demonstrated target engagement and elicited appropriate pharmacodynamic changes in these patients.

### Example 5 - Inhibition of Cytokine Storms by PTC299 - BioMAP Profiling

DHODH inhibitors inhibit cytokine storms that significantly contribute to pulmonary pathology, organ failure, and death (Yoshikawa 2009, Xiong 2020). Specifically, levels of IL-2, IL-7, IL-10, GCSF, IP-10, MCP1, MIP1A, and TNFa were found to be higher in COVID-19 patients in the intensive care unit (ICU) than in those patients not in the ICU thus indicating that detection of elevated amounts of these cytokines may be predictive markers of severe disease (Huang 2020).

The effect of PTC299 on cytokine production was assessed using BioMAP profiling (BioSeek, now part of Eurofins). The BioMAP system is comprised of complex co-cultures of primary peripheral blood mononuclear cells (PBMCs) with other cell types including B cells, fibroblasts, and endothelial cells allowing for the measurement of physiologically relevant biomarker readouts of the activity of a compound. Using this system, cytokine production was evaluated in the following models of primary cell disease models:
Peripheral blood mononuclear cells (PBMCs) plus venular endothelial cells (sAg)
PBMCs plus B cells (BT)
PBMCs plus fibroblasts (HDFSAg)
T_{H2} cells plus venular endothelial cells (/TH2).

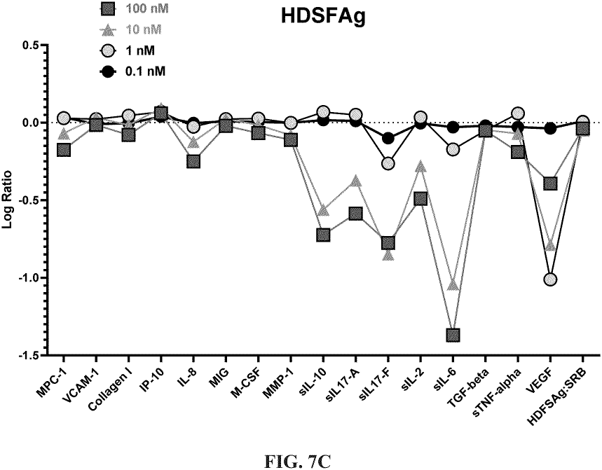

Study results are shown in Table 4, below, and in FIGS. 7A-7E.

TABLE 4

Summary of PTC299 Inhibition of Cytokines from BioMAP Profile

| Cytokine | Co-culture system | Dose (nM) with p<0.01 | % Inhibition |
|---|---|---|---|
| MCP-1 | SAg | 10, 100 | 31, 45 |
| CD40 | SAg | 100 | 29 |
| IL-8 | SAg | 10, 100 | 27, 29 |
| Proliferation | SAg | 100 | 23 |
| sIgG | BT | 10, 100 | 97, 98 |
| sIL- 17A | BT | 10, 100 | 68, 83 |
| s-IL- 17F | BT | 10, 100 | 58, 74 |
| s-IL-6 | BT | 10, 100 | 53, 72 |
| sTNFa | BT | 10, 100 | 49, 66 |
| B cell proliferation | BT | 100 | 31 |
| MCP-1 | HDFSAg | 100 | 33 |
| IL-8 | HDFSAg | 10, 100 | 25, 44 |
| MMP-1 | HDFSAg | 100 | 23 |
| sIL-10 | HDFSAg | 10, 100 | 73, 81 |
| sIL- 17A | HDFSAg | 10, 100 | 57, 74 |
| sIL-17F | HDFSAg | 1, 10, 100 | 46, 86, 83 |
| sIL-2 | HDFSAg | 10, 100 | 47, 68 |
| sIL-6 | HDFSAg | 1, 10, 100 | 33, 91, 96 |
| sTNFa | HDFSAg | 100 | 35 |
| sVEGF | HDFSAg | 1, 10, 100 | 90, 84, 60 |
| MCP-1 | /TH2 | 10, 100 | 21,25 |
| sIL-17 | /TH2 | 100 | 23 |

Abbreviations in Table 4: BCR, B-cell receptor; BT System, co-culture of CD19+ B cells and PBMC that utilizes BCR stimulation and sub-mitogenic TCR stimulation; HDFSAg system, co-culture of human primary dermal fibroblasts and PBMC that is stimulated with sub-mitogenic TCR levels; IL, interleukin; PBMC, Peripheral Blood Mononuclear Cell; SAg system, co-culture of endothelial cells and PBMC stimulated with mitogenic levels of TCR ligands; TCR, T-cell receptor; /TH2 system, co-culture of endothelial cells and Th2 blasts stimulated with TCR ligands and cytokines; Th2, T helper type 2; VEGF, vascular endothelial growth factor.

PTC299 was found to be a potent inhibitor of immunomodulatory and inflammation-related activities in the assay described immediately above. Compared with vehicle control, PTC299 resulted in:
Decreases in immunomodulatory-associated molecules: CD40, sIgG, sIL-17A, sIL-17F, sIL-6, sIL-2, sIL-10;
Decreases in inflammation-related molecules: MCP-1, IL-8, and sTNFa.

The ability of PTC299 to decrease the levels of these molecules is relevant to COVID-19 as many of them have been shown to be elevated in hospitalized patients infected with SARS-CoV-2 (Chen 2020a, Huang 2020, Wang 2020b, Xu 2020). Interestingly, clear evidence exists that IL-6 peak levels are associated with severity of pulmonary complications (Russell 2020).

### Example 6 - Inhibition of Cytokine Storms by PTC299 - Cytokine Release from IL-2/CD3 and CD28 Antibody Stimulated PBMCs

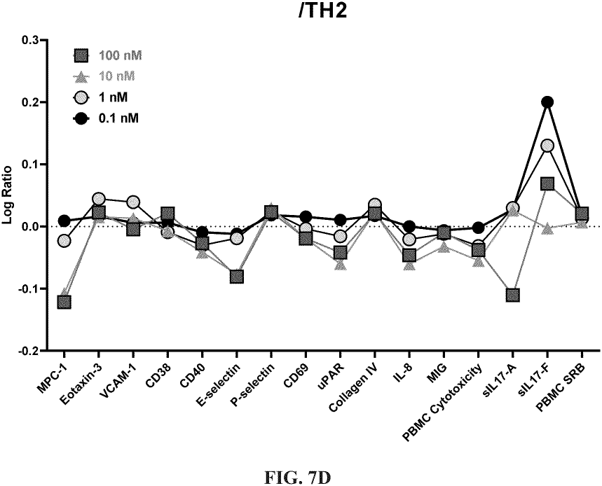

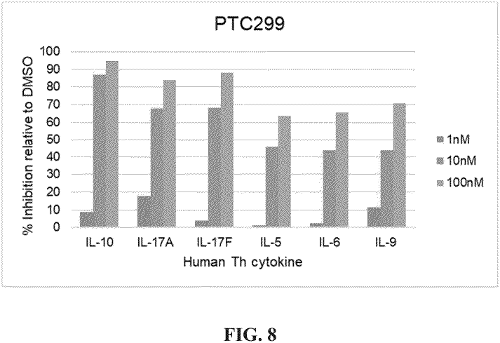

Another study assessed cytokine release from IL-2/ CD3 and CD28 antibody stimulated PBMCs after 48 hours of incubation. Cytokine analysis was done using a multiplex assay kit. Levels of multiple cytokines were decreased with PTC299 treatment (FIG. 8), further indicating that PTC299 can reduce cytokine expression. The inhibition of at least IL-17A and IL-17F was specific to DHODH inhibition as both PTC299 and brequinar inhibited production of IL-17A and IL-17F, while the inactive R-enantiomer PTC-371 did not (FIGS. 9A and 9B, respectively).

### Example 7 - Non-Clinical Safety Studies

In vitro and in vivo safety pharmacology studies with PTC299 have demonstrated a favorable safety profile. Based on the safety pharmacology studies and results of electrocardiograms and blood pressure measurements made during 7- and 28-day toxicity studies in dogs, PTC299 is considered unlikely to cause serious adverse effects on the central nervous, cardiovascular, and respiratory systems (Study QFE00014). PTC299 was negative in a bacterial mutagenicity (Ames) test, a genotoxicity study to investigate chromosomal aberration assay in Chinese hamster ovary cells, and in an in vivo rat micronucleus assay.

A comprehensive toxicology program was conducted for PTC299 that included genetic toxicology studies, single- and repeat-dose (7- and 28-day; with 2-week recovery periods in the 28-day studies) studies in rats and dogs. No PTC299-related mortality was observed in the toxicology studies. Rats and dogs dosed through 28 days exhibited no histopathologic findings in the liver, and no increases in alanine aminotransferase, aspartate aminotransferase, and total bilirubin were observed.

### Example 8 - Clinical Studies

### Study Design and Rationale

The study of PTC299 in COVID-19 is an integrated phase ⅔ trial that is designed to evaluate the efficacy and safety of hospitalized adult patients with confirmed pneumonia, diagnosed with COVID-19 and who do not require mechanical ventilation. The complete trial is a randomized, placebo-controlled, multicenter, 28-day study. The study has 2 stages, the first stage of which has been completed. A smaller cohort of subjects, 40 subjects, were enrolled in the first stage followed by an interim safety analysis before enrolling the larger cohort in the second stage. A larger cohort of subjects, the original 40 subjects from stage 1, and an 55 additional subjects from stage 2 have been enrolled so far.

Eligible subjects are hospitalized adult patients (≥18 years of age) diagnosed with SARS-CoV-2 infection and who are not on mechanical ventilation. Subjects are required to have an SpO2 <94% on room air, a respiratory rate >24 breaths/minute or cough, and to have radiographic infiltrates by imaging (chest x-ray, computed tomography scan, or an equivalent test) indicating lung involvement, prior to enrollment.

Subjects are randomized 1:1 to either PTC299 treatment plus the SOC or placebo plus the SOC alone. Standard of care is defined as the SOC per local written policies or guidelines but excludes the use of steroids, with the exception of dexamethasone. The total study population will be about 380 subjects with 190 subjects in each cohort.

The primary endpoint of the study has been and still is:

Time from randomization to respiratory improvement, defined as SpO2 ≥ 94% on room air, sustained until discharge from the hospital or the end of the study Day 28).

Secondary endpoints include the proportion of subjects requiring invasive ventilation at any point during the study, proportion of subjects needing supplemental oxygen or non-invasive ventilation, time to defervescence in subjects presenting with fever at enrollment, time from randomization to respiratory rate <24 breaths/minute on room air, time from randomization to cough reported as mild or absent (on a scale of severe, moderate, mild, absent, in those with cough at enrollment rated severe or moderate), Time from randomization to dyspnea reported as mild or absent (on a scale of severe, moderate, mild, absent, in those with dyspnea at enrollment rated as severe or moderate) attenuation of immune-responses, reduction in viral load, duration of hospitalization, mortality at Day 28, and overall safety profile.

### Dosing of PTC299

In this study, subjects have been or will be dosed for a total of 14 days and followed for an additional 14 days without dosing. Subjects have or will receive oral tablets containing PTC299 or matched placebo 200 mg twice daily (BID) (morning and evening) on Days 1 to 7 and PTC299 or matched placebo 50 mg once daily (QD) on Days 8 to 14. Subjects will subsequently be followed from Day 15 to 2828, and there will be a telephone call at Day 60 to assess AEs, SAEs, and death.

The rationale for this dosing is based on the following:
The dosing regimen utilizes results from PTC’s extensive clinical, safety, and clinical pharmacology experience with the capsule formulation of PTC299 that was used in prior solid tumor clinical studies.
   The tablet formulation has approximately 40% relative bioavailability compared with the capsule when administered with food.
The 200 mg BID dosing for 7 days have been tested previously in healthy volunteers in study PTC299-ONC-002-HV with no serious adverse events. The dose-related hepatotoxicity associated with PTC299 was observed at higher doses and longer treatment durations (>85 days) than what will be used in this study.
The dosing regimen is designed to maximize the therapeutic effect. The dosing of 200 mg BID for the first 7 days will raise the exposure of the drug quickly to accommodate the need for rapid onset of PTC299 activity.. The dosing of 50 mg QD for the second 7 days will prevent the exposure from substantially decrease.
   The plasma concentration of drug following 7 days of 200 mg BID dosing is similar to that which demonstrated inhibition of DHODH in the clinical study PTC299-ONC-007-NF2 (see Example 4, above).
Prior clinical studies have also demonstrated that the pharmacokinetics (PK) of PTC299 is similar between cancer patients and healthy volunteers, suggesting similar PK would be anticipated in COVID-19 patients.

### Experimental Support for Dosing

The PK of the 200 mg BID dosed for 7 days and 50 mg for 7 days was evaluated in a phase 1, escalating, multiple dose, safety, tolerability, and PK study, PTC299-ONC-002-HV (N=32; n=24 received PTC299 and 8 received placebo). Importantly, no SAEs occurred during the study.

Study PTC299-ONC-002-HV had 2 stages; in Stage 1, doses ranged from 0.3 to 1.2 mg/kg BID, and in Stage 2, a 1.6 mg/kg TID dose was administered. Patients received the capsule formulation of PTC299 for 7 days in both stages. Dose of 200 mg tablet is approximately 1.2 mg/kg capsule whereas 50 mg tablet is approximately 0.3 mg/kg of capsule formulation.

The PK of PTC299 in healthy subjects in study PTC299-ONC-002-HV is summarized in Table 5.

TABLE 5

Summary of PTC299 Pharmacokinetics in Healthy Volunteers when Administered with the Capsule Formulation (~ 2.5x of the Bioavailability of the Tablet Formulation)

| Study | Dose (mg/kg) | Dose Frequency | Day | PTC299 | | | |
|---|---|---|---|---|---|---|---|
| PTC299-ONC-002-HV | | | | Tmax (h) | T_{l/2} (h) | Cmax (µg/mL | AUC_{O-4} (µg_{*}hr/mL) |
| | 0.3 | BID | 1 | 4.0 | NA | 0.28 | 4.31 |
| | 0.6 | | | 3.0 | | 0.71 | 10.1 |
| | 1.2 | | | 3.5 | | 1.14 | 18.0 |
| | 1.6 | TID | | 3.0 | | 1.67 | 37.1 |
| | 0.3 | BID | 7 | 3.5 | 164 | 0.51 | 8.44 |
| | 0.6 | | | 4.0 | 210 | 1.11 | 18.6 |
| | 1.2 | | | 4.0 | 228 | 2.11 | 32.9 |
| | 1.6 | TID | | 3.0 | 225 | 3.53 | 78.6 |

Abbreviations: AUC₀₋₄, area under the concentration curve from hour 0 to 4; Cₘₐₓ, maximal concentration; T_{½}, half-life; Tₘₐₓ, time to maximal concentration.

Prior clinical and pre-clinical data indicate the level of PTC299 that was expected to be achieved with 7 days of 200 mg BID dosing will result in meaningful inhibition of DHODH.

As indicated in Example 4, dosing for 8 or 16 weeks with 100 mg BID of the capsule formulation in study PTC299-ONC-007-NF2 resulted in an increase in the plasma level of DHO and restoration of VEGF levels in patients with neurofibromatosis type 2, consistent with inhibition of DHODH. In the same study, the 8-week plasma level at 4 hours post-dose was similar to the Cₘₐₓ observed in PTC299-ONC-002-HV (1.9 µg/mL vs. 2.11 µg/mL, respectively). These results suggest that the proposed 200 mg BID dosing for the proposed study will be effective in inhibiting DHODH activity.

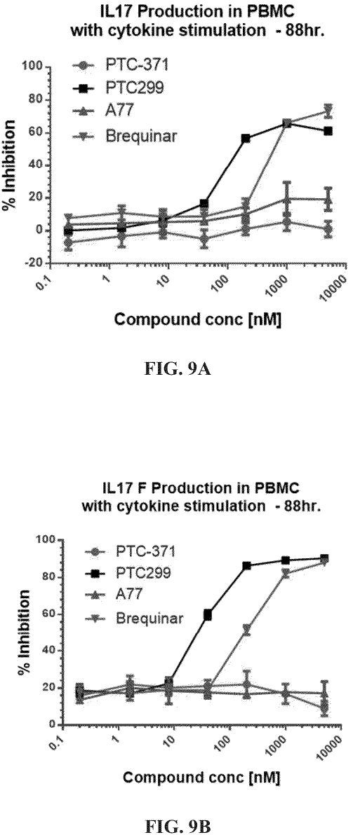

The dosing regimen used in the current study was also supported by experiments performed in Rhesus monkeys. Monkeys were given a single oral 10 mg/kg dose of PTC299. The DHO plasma level increased after dosing, peaked at 24 hours and then decreased, as illustrated in FIG. 10. The AUC for PTC299 was 22.5 µg*hr/mL. The peak DHO level was delayed relative to the peak concentration of PTC299 (24 hours vs. 2 hours), as shown in Table 6 below. The AUC for PTC299 is consistent with that observed in the healthy volunteer study PTC299-ONC-002-HV indicating this exposure would result in inhibition of DHODH and supports the use of the 200 mg BID dosing regimen in the current study.

TABLE 6

Summary of PK and PD Parameters

| Parameter | PTC299 | DHO |
|---|---|---|
| AUC (µg*hr/mL) | 22.5 | 71.4 |
| Cₘₐₓ (µg/mL) | 2.51 | 2.48 |
| Tₘₐₓ (hr post-dose) | 2 | 24 |
| Half-life (hr) | 33 | 10 |

Abbreviations: AUC, area under the curve: DHO, dihydroorotate

### PTC299 200 Mg Tablet BID for 7 Days Is Expected to Be Tolerated in Patients

The AUC at Day 7 in the study is expected to be approximately 32.9 ug*h/mL, which was observed in healthy subjects from study PTC299-ONC-002-HV who received 1.2 mg/kg capsule BID for 7 days (Table 5). No SAEs were reported in PTC299-ONC-002-HV.

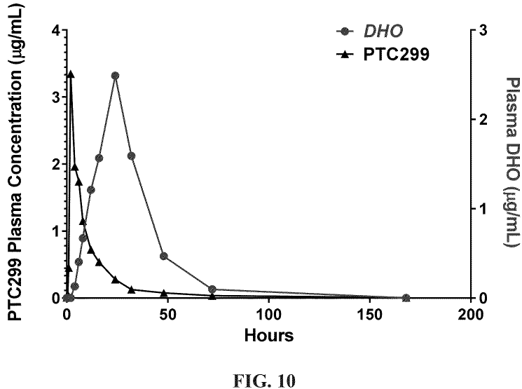

The PK characteristics of PTC299 and DILIsym modeling suggest that limited PTC299 exposure at 32.9 ug*h/mL is unlikely to result in hepatotoxicity, as shown in FIGS. 11A and 11B. In clinical studies, O-desmethyl PTC299 had a systemic exposure of about 30% to 50% that of PTC299.

FIGS. 11A and 11B show the following. Simulated desmethyl PTC299 Cₘₐₓ (a) and AUC₀₋₂₄ₕᵣ (b) on day 112 versus maximum ALT in three selected individuals with varying sensitivity from the SimCohorts v4A-1-Multi16; ID_2 (blue, the most sensitive individual), ID_10 (red, the individual with intermediate sensitivity), and ID_15 (green, the least sensitive individual). Simulations were performed with 100 - 330 mg/dose capsule BID PTC299 dosing for 16 weeks. Light and dark pink shades represent max ALT > 1X and 3X ULN, respectively. Black arrows represent the minimum desmethyl PTC299 Cₘₐₓ or AUC₀₋₂₄ₕᵣ that showed max ALT > 1X or 3X ULN among the simulated individuals. Source: Project Work Orders (PWO) 002,003, and 004: Assessing the Optimal Dose and Liver Safety of PTC299 with the DILIsym Software Representation of PTC299 Developed During PWO 001; Oct. 11, 2017; FIGS. 30a and 30b.

It was anticipated that elimination would not be saturated with 50 mg tablet QD after 7 days of 200 mg tablet BID dosing. This is supported by results from the ongoing AML open-label, non-randomized, phase 1b study, PTC299-HEM-001-LEU. In the AML study, steady state was reached at approximately Day 15 after administration of an 80 mg QD loading dose of the tablet formulation of PTC299 for 7 days followed by a 40 mg maintenance dose (cohort 2).

The Cₘₐₓ after 200 mg tablet BID for seven 7 days is anticipated to be about 2.11 ug/mL and is not expected to cause any safety concern, as similar plasma concentrations were observed in the patients who were treated with PTC299 capsule for >40 days.

### Safety Monitoring

Subjects were monitored closely for adverse events (AEs) and laboratory abnormalities during the study. An independent unblinded data and safety monitoring board (DSMB) monitored ongoing results to ensure subject well-being and safety as well as study integrity. The DSMB could have made recommendations about early study closure or changes to study arms. However, no such recommendations were made.

The safety profile of patients in this proposed study was anticipated to be different from that previously observed in solid tumor populations in which higher doses of PTC299 were employed. The risk associated with PTC299 is primarily dose-related hepatotoxicity that was observed at higher doses and longer treatment durations than those that will be used in this study.

Specific measures were undertaken to identify the risk of hepatotoxicity early in subjects and to manage drug exposure if that risk is confirmed. Specifically, comprehensive monitoring of liver function tests (LFTs) were performed, including measurement of ALT, AST, total bilirubin (Tbili), and alkaline phosphatase plasma levels weekly during treatment.

Careful monitoring ensured limited accumulation of either PTC299 or the O-desmethyl PTC299 metabolite and that the mean AUC from 0 to 24 hours (AUC₀₋₂₄ₕᵣ) values in treated subjects remain below levels associated with hepatotoxicity.

The DSMB reviewed the results of the blinded safety study involving the first 40 subjects, and approved the plan to proceed to the next stage of the study.

### Tablet Information

PTC299 tablets for oral administration will be provided in dosage strengths of 10 and 50 mg. PTC had a sufficient supply of PTC299 10 mg and 50 mg tablets for the proposed integrated phase ⅔ study.

### Example 9 - Inhibition of Replication of SARS-CoV-2 in Vero Cells - Viral Titer

Four plates of Vero cells were pretreated with the concentration of PTC299 and PTC299 with uridine as indicated in Table 7, below.

TABLE 7

PTC299 Concentration and Treatment

| | |
|---|---|
| PTC299 Concentration | Treatment ID |
| 0.25, 0.5, 1, 0 µm | 18 h pretreatment with PTC299 |
| 0.25, 0.5, 1, 0 µm | 18 h pretreatment with PTC299 and uridine |
| 0.25, 0.5, 1, 0 µm | 2 h pretreatment with PTC299 |
| 0.25, 0.5, 1, 0 µm | 2 h pretreatment with PTC299 and uridine |

Abbreviations: h, hours

After incubation at 37° C. in CO₂ overnight, media was removed, and cells were inoculated with SARS-CoV-2 at a multiplicity of infection (MOI) of 0.05. After 1 hour of incubation at 37° C. in CO₂, wells were washed 3 times with dilution media and 1 mL of media containing the indicated compound doses was added back to each well. Cells were incubated at 37° C. in CO₂ and samples collected at 0, 16, and 24 hours post-infection. Collected timepoint media was replaced with an equal amount of 1x compound or dilution media. Samples were stored at -80° C. until the day of analysis. The SARS-CoV-2 titer in Vero cells via 50% tissue culture infectious dose assay (TCID₅₀) was performed for each sample collected at 0, 16, and 24 hours post infection. Viral titer was evaluated by collecting the medium from the infected cell cultures and calculating the concentration of PTC299 required for 50% infectivity (the 50% tissue culture infections dose assay or TCID₅₀).

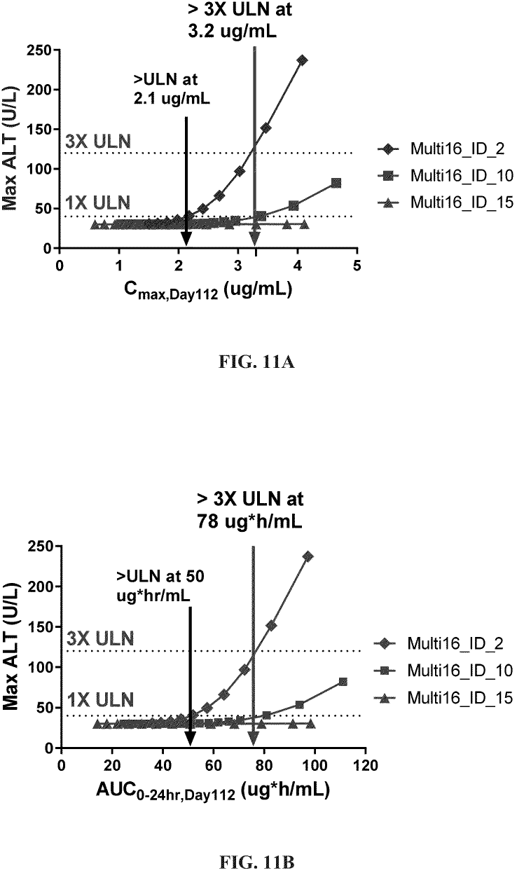

Results of the assay are illustrated in FIGS. 12A-12D. FIG. 12A shows viral titer at each dose after 18 hours preincubation. FIG. 12B shows viral titer at each dose after 18 hours of preincubation with uridine. FIG. 12C shows viral titer after 2 hours pretreatment with PTC299, and FIG. 12D shows viral titer after 2 hours pretreatment with PTC299 and uridine.

Pre-incubation of Vero cells with PTC299 at 250, 500, and 1000 nM for 2 hours prior to viral infection reduced viral replication at all dose levels tested by about 3 orders of magnitude after 24 hours (FIG. 12A). To determine whether the effect of PTC299 is a direct consequence of DHODH inhibition, excess uridine (100 uM) was added (uridine obviates the need for de novo pyrimidine nucleotide biosynthesis by DHODH). Addition of excess uridine overcame inhibition of SARS-CoV-2 replication by PTC299, as illustrated in FIG. 12B. That result is consistent with the drug inhibiting DHODH to block viral replication. PTC299 also inhibited viral replication when cells were preincubated for 18 hours with or without uridine, as illustrated in FIGS. 12C and 12D. Uridine was added to the cell cultures infected with SARS-CoV-2 in this Example to confirm that the result described above was due to DHODH inhibition. Uridine addition bypasses the requirement of de novo pyrimidine synthesis, allowing viral replication. The addition of uridine negates the need for DHODH and the de novo pyrimidine pathway and is consistent with PTC299 acting through DHODH inhibition to block viral replication.

### Example 10 - Inhibition of Replication of SARS-CoV-2 in Vero Cells - Staining

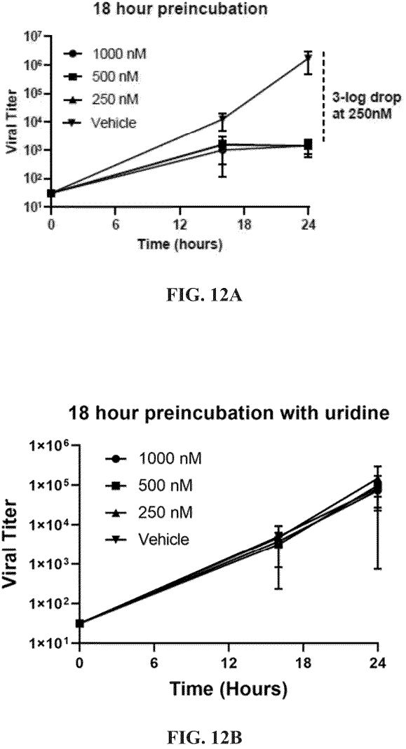

Five cultures of Vero cells were prepared, four of which were infected with SARS-CoV-2 and treated with either DMSO, 30 nM PTC299, 300 nM PTC299, or 3 µM PTC299 and then stained with DAPI. The stained cells are shown in FIGS. 13A-13E. The cells in FIG. 13A are uninfected; the cells in FIG. 13B were treated with DMSO, and while the cells in FIGS. 13C-13D were treated with 30 nM PTC299, 300 nM PTC299, or 3 µM PTC299, respectively. Viral levels were determined by staining cells using the N-protein antibody, as reflected by the green fluorescence. Increasing the PTC299 concentration led to greater reductions of SARS-CoV02 compared with untreated controls. However, the viability of the cells, as reflected by the blue stain, remained unaffected, showing that PTC299 was not cytotoxic.

### Example 11 - Additional Study of Inhibition of Replication of SARS-CoV-2 in Vero Cells

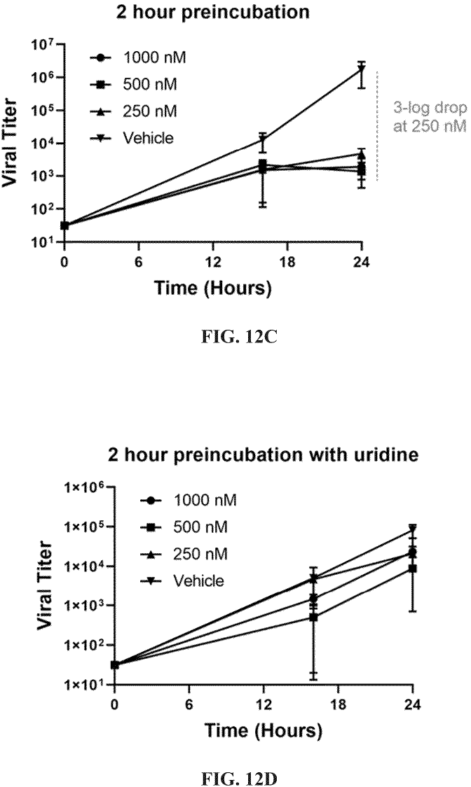

Results of an additional study of SARS-CoV2 infected Vero Cells either untreated or treated similarly to Example 8 above, with varying amounts (1, 0.5, or 0.25 µM) of PTC299 are shown in FIGS. 14A and 14B . FIG. 14A shows the results produced with two hours of pre-treatment with PCT299, while FIG. 14B shows results following eighteen hours of pre-treatment with PTC299. Similar to Example 8, at 24 hours post infection, a three log drop in titer was observed in cells treated with PTC299 as compared to untreated cells.

### Example 12 - Inhibition of Replication of SARS-CoV-2 in Vero Cells -Determination of EC₅₀ and EC₉₀

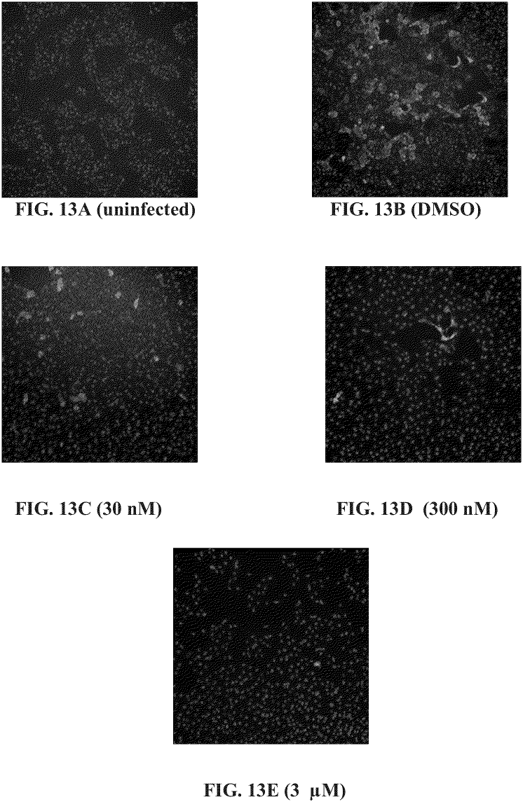

The 50% and 90% effective concentrations (EC₅₀ and EC₉₀, respectively) were determined by conducting an extended dose response study, as follows. Vero cells were preincubated with PTC299 at 0.003, 0.01, 0.10, 0.30, 1.0, 3.0, 10, 20, 30, 40, 50, 100, 250, 500, 750, and 1000 nM prior to addition of SARS-CoV-2. A plot of the assay results is shown in FIG. 15. The EC₅₀ and EC₉₀ values were determined to be 2.6 nM and 53 nM, respectively, which is consistent with the findings of the immunofluorescence experiments described in Example 9, above.

### Example 13 - Inhibition of Replication of SARS-CoV-2 in Vero Cells -Determination of CC₅₀

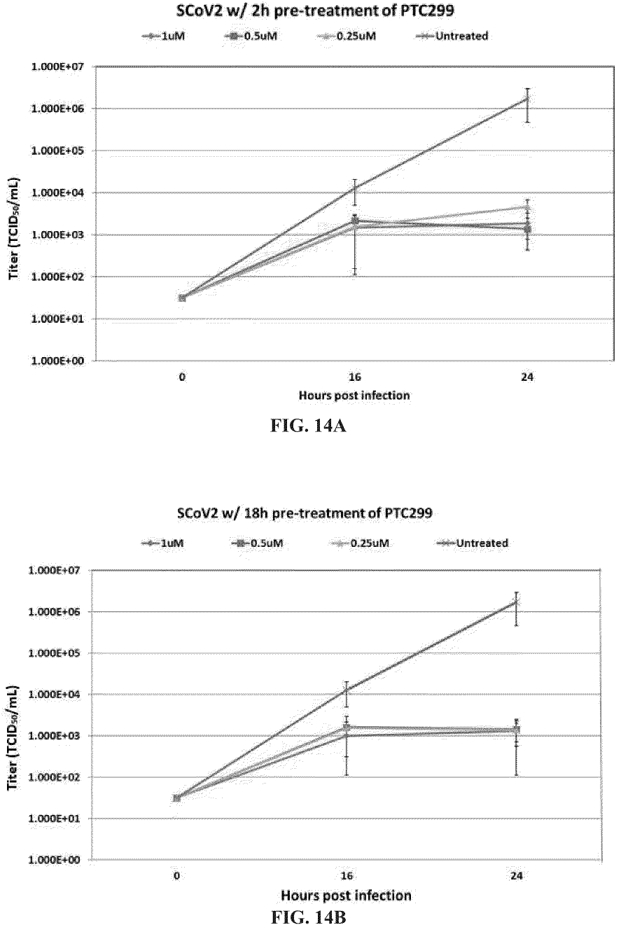

In parallel with the studies described in Examples 8-10, the concentration of compound necessary to reduce cell viability by 50% (cytotoxic concentration; CC₅₀) was determined by measuring ATP levels. The selective index was established, as follows: Vero cells were inoculated with SARS-CoV-2 (USA-WA1/2020) at a MOI of 0.05. Viral titer was measured by collecting the medium from the infected cells and assessing the concentration required for 50 % infectivity of Vero cells (the 50 % tissue culture infectious dose assay or TCID₅₀). Cytotoxicity was evaluated by measuring intracellular ATP (adenosine triphosphate) levels after 48 hours in culture. Data was plotted as the mean and standard deviation of 3 independent replicates. At the highest concentration of PTC299 tested (10 µM), the CC₅₀ was determined to be > 10 µM (FIG. 16), indicating that, similar to the immunofluorescence experiments described above, PTC299 reduces viral load with little cell death.

### Example 14 - Combination of PTC299 With Remdesivir

### Materials and Methods

Vero cells (CCL-81, American Type Culture Collection [ATCC]) were maintained in Dulbecco’s modified eagle medium (Hyclone) supplemented with 10% fetal bovine serum (Gibco) and 1% penicillin and streptomycin solution (Hyclone). The SARS-CoV-2 USA-WA1/2020 was provided by the World Reference Center for Emerging Viruses and Arboviruses and was originally obtained from the Centers for Disease Control and Prevention.

Three 12-well plates of Vero cells (2×105 cells per well) were pretreated with PTC299 and/or remdesivir for 4 hours at 37° C. Following incubation, cells were inoculated with multiplicity of infection 0.05 SARS-CoV-2 premixed with the appropriate drug concentration in growth medium. After 1 hour of incubation at 37° C. in 5% CO2, wells were washed 3 times with dilution medium and 1 mL of medium containing the indicated compound concentration was added back to each well. Cells were incubated at 37° C. and samples collected at 0, 16, and 24 hours post infection. Collected timepoint medium was replaced with an equal amount of 1x compound or dilution medium. Samples were stored at -80° C. until the day of analysis. The SARS-CoV-2 titer in Vero cells was determined using a 50% tissue culture infectious dose assay (TCID₅₀) for each sample collected at 0, 16, and 24 hours post infection.

### Results

PTC299 and remdesivir were tested individually or in combination at 2 concentrations. Vero cells were pretreated with the appropriate compound for 4 hours prior to infection with SARS-CoV-2. Samples were collected at 0, 16, and 24 hours post infection and used to determine titer in the TCID₅₀ assay.

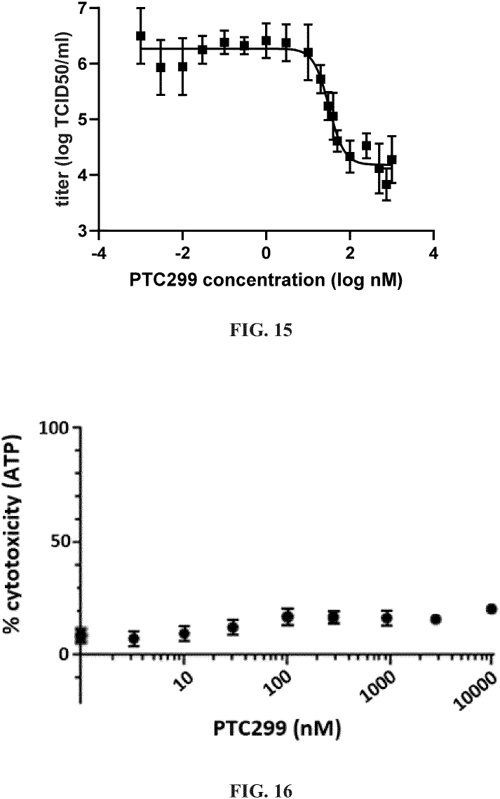

Remdesivir at a concentration of 5 µM caused a reduction in titer of 86% relative to untreated control cells at 24 hours post infection but did not cause a reduction in titer at 2.5 µM (FIG. 17). PTC299 reduced the SARS-CoV-2 titer by 98% and 96% at concentrations of 500 and 250 nM, respectively, when sampled 24 hours post infection (FIG. 17). FIG. 17 specifically shows that PTC299 and remdesivir both reduce the replication of SARS-CoV-2 in Vero cells, and in a concentration dependent manner, when used separately to treat infected cells.

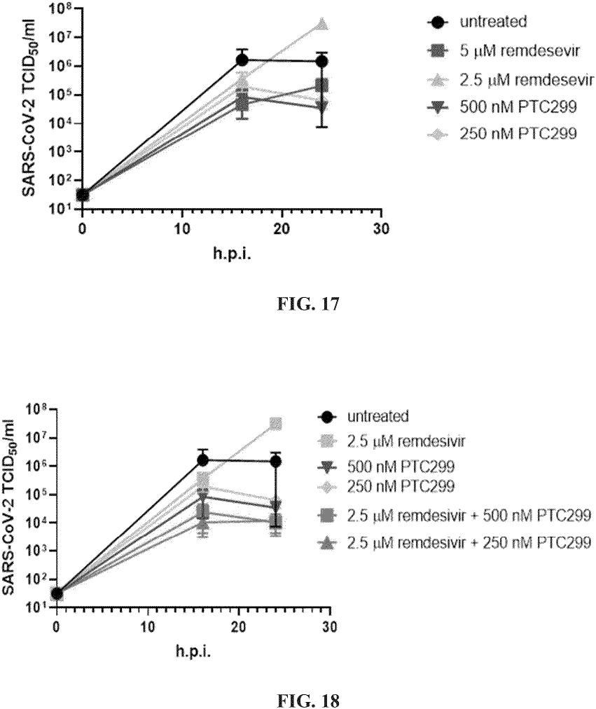

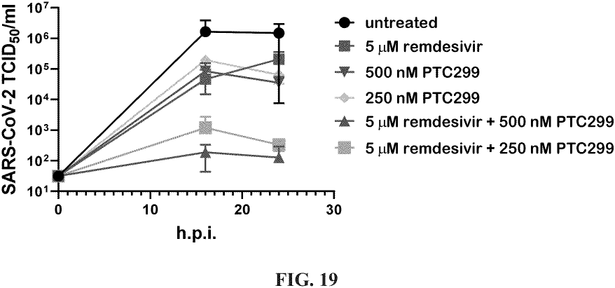

While at a concentration of 2.5 µM remdesivir was not effective in reducing vital titer when administered alone, it provided a modest increase of antiviral activity when administered in combination with PTC299, reducing the viral titer by 99.3% with 500 nM PTC299 and 99.2% with 250 nM PTC299 when sampled at 24 hours post infection (FIG. 18). However, when the remdesivir concentration was increased to a concentration which did show standalone antiviral activity, a significant increase in virus inhibition was observed. At 24 hours post infection, treatment with 5 µM remdesevir in combination with 500 or 250 nM PTC299 reduced viral titers by >99.99% and 99.98%, respectively (FIG. 19).

### Conclusions

Each alone, PTC299 and remdesivir demonstrated antiviral activity against SARS-CoV-2 replication in Vero cells. PTC299 was much more potent, with 250 nM PTC299 demonstrating greater reduction of viral titer than did a 20-fold higher dose of remdesivir (5 µM). PTC299 has been demonstrated to inhibit cellular pyrimidine nucleotide biosynthesis, while remdesivir is a purine nucleotide analog. This study was undertaken to determine whether a combination treatment with these 2 antiviral agents can have an enhanced effect on the inhibition of viral production.

The antiviral activity of the single agent treatments against SARS-CoV-2 was established by FIG. 17. The low dose of remdesivir tested, 2.5 µM, did not have antiviral activity alone, but did increase PTC299 activity when added in combination (FIG. 18). While PTC299 and 5 µM remdesivir (FIG. 19) demonstrated antiviral activity as single agents, combination treatment resulted in much greater inhibition of SARS-CoV-2 replication.

### REFERENCES

Aarava® Package insert; Summary of Product Characteristics (SPC).

Aarava® Package insert; Summary of Product Characteristics (SPC).

Aubagio® Package insert.

Bar-Or, A, Pachner, A, Menguy-Vacheron, F, Kaplan, J and Wiendl, H. Teriflunomide and its mechanism of action in multiple sclerosis. Drugs 2014;74(6):659-674.

Breedveld, FC and Dayer, JM. Leflunomide: mode of action in the treatment of rheumatoid arthritis. Annals of the rheumatic diseases 2000;59(11):841-849.

Cao, B, Wang, Y, Wen, D, Liu, W, Wang, J, Fan, G, et al. A Trial of Lopinavir-Ritonavir in Adults Hospitalized with Severe Covid-19. N Engl J Med 2020.

CEBM. (2020). “Global Covid-19 Case Fatality Rates.” Retrieved Mar. 30, 2020, from https://www.cebm.net/covid-19/global-covid-19-case-fatality-rates/.

Chen, N, Zhou, M, Dong, X, Qu, J, Gong, F, Han, Y, et al. Epidemiological and clinical characteristics of 99 cases of 2019 novel coronavirus pneumonia in Wuhan, China: a descriptive study. Lancet 2020a;395(10223):507-513.

Chen, S, Ding, S, Yin, Y, Xu, L, Li, P, Peppelenbosch, MP, et al. Suppression of pyrimidine biosynthesis by targeting DHODH enzyme robustly inhibits rotavirus replication. Antiviral Res 2019;167:35-44.

Chen, SF, Perrella, FW, Behrens, DL and Papp, LM. Inhibition of dihydroorotate dehydrogenase activity by brequinar sodium. Cancer Res 1992;52(13):3521-3527.

Chen, ZM, Fu, JF, Shu, Q, Chen, YH, Hua, CZ, Li, FB, et al. Diagnosis and treatment recommendations for pediatric respiratory infection caused by the 2019 novel coronavirus. World J Pediatr 2020b.

Cheng, ZJ and Shan, J. 2019 Novel coronavirus: where we are and what we know. Infection 2020.

Cheung, NN, Lai, KK, Dai, J, Kok, KH, Chen, H, Chan, KH, et al. Broad-spectrum inhibition of common respiratory RNA viruses by a pyrimidine synthesis inhibitor with involvement of the host antiviral response. J Gen Virol 2017;98(5):946-954.

Chien, JY, Hsueh, PR, Cheng, WC, Yu, CJ and Yang, PC. Temporal changes in cytokine/chemokine profiles and pulmonary involvement in severe acute respiratory syndrome. Respirology 2006;11(6):715-722.

Coronavirus Outbreak. Available at: https://www.worldometers.info/coronavirus/. Accessed Mar. 26, 2020.

Diedrichs-Mohring, M, Niesik, S, Priglinger, CS, Thurau, SR, Obermayr, F, Sperl, S, et al. Intraocular DHODH-inhibitor PP-001 suppresses relapsing experimental uveitis and cytokine production of human lymphocytes, but not of RPE cells. J Neuroinflammation 2018;15(1):54.

Duley, JA, Henman, MG, Carpenter, KH, Bamshad, MJ, Marshall, GA, Ooi, CY, et al. Elevated plasma dihydroorotate in Miller syndrome: Biochemical, diagnostic and clinical implications, and treatment with uridine. Mol Genet Metab 2016;119(1-2):83-90.

Dunn, MC, Knight, DA and Waldman, WJ. Inhibition of respiratory syncytial virus in vitro and in vivo by the immunosuppressive agent leflunomide. Antivir Ther 2011;16(3):309-317.

FDA. (2009). “FDA Dili Guidance July2009.” from https://www.Fda.Gov/downloads/drugs/.../guidances/ucm174090.Pdf.

Hall, AP, Elcombe, CR, Foster, JR, Harada, T, Kaufmann, W, Knippel, A, et al. Liver hypertrophy: a review of adaptive (adverse and non-adverse) changes--conclusions from the 3rd International ESTP Expert Workshop. Toxicol Pathol 2012;40(7):971-994.

Hoffmann, HH, Kunz, A, Simon, VA, Palese, P and Shaw, ML. Broad-spectrum antiviral that interferes with de novo pyrimidine biosynthesis. Proc Natl Acad Sci U S A 2011;108(14):5777-5782.

Huang, C, Wang, Y, Li, X, Ren, L, Zhao, J, Hu, Y, et al. Clinical features of patients infected with 2019 novel coronavirus in Wuhan, China. Lancet 2020;395(10223):497-506.

Jin, YH, Cai, L, Cheng, ZS, Cheng, H, Deng, T, Fan, YP, et al. A rapid advice guideline for the diagnosis and treatment of 2019 novel coronavirus (2019-nCoV) infected pneumonia (standard version). Mil Med Res 2020;7(1):4.

Kut, C, Mac Gabhann, F and Popel, AS. Where is VEGF in the body? A meta-analysis of VEGF distribution in cancer. British journal of cancer 2007;97(7):978-985.

Lewis, TA, Sykes, DB, Law, JM, Muñoz, B, Rustiguel, JK, Nonato, MC, et al. Development of ML390: a human DHODH inhibitor that induces differentiation in acute myeloid leukemia. ACS medicinal chemistry letters 2016;7(12):1112-1117.

Li, Q, Guan, X, Wu, P, Wang, X, Zhou, L, Tong, Y, et al. Early Transmission Dynamics in Wuhan, China, of Novel Coronavirus-Infected Pneumonia. N Engl J Med 2020;382(13):1199-1207.

Li, Y, Wang, S, Wang, Y, Zhou, C, Chen, G, Shen, W, et al. Inhibitory effect of the antimalarial agent artesunate on collagen-induced arthritis in rats through nuclear factor kappa B and mitogen-activated protein kinase signaling pathway. Transl Res 2013;161(2):89-98.

Liu, K, Fang, YY, Deng, Y, Liu, W, Wang, MF, Ma, JP, et al. Clinical characteristics of novel coronavirus cases in tertiary hospitals in Hubei Province. Chin Med J (Engl) 2020a.

Liu, Y, Yang, Y, Zhang, C, Huang, F, Wang, F, Yuan, J, et al. Clinical and biochemical indexes from 2019-nCoV infected patients linked to viral loads and lung injury. Sci China Life Sci 2020b;63(3):364-374.

Lolli, ML, Sainas, S, Pippione, AC, Giorgis, M, Boschi, D and Dosio, F. Use of human Dihydroorotate Dehydrogenase (hDHODH) Inhibitors in Autoimmune Diseases and New Perspectives in Cancer Therapy. Recent Pat Anticancer Drug Discov 2018;13(1):86-105.

Lucas-Hourani, M, Dauzonne, D, Jorda, P, Cousin, G, Lupan, A, Helynck, O, et al. Inhibition of pyrimidine biosynthesis pathway suppresses viral growth through innate immunity. PLoS Pathog 2013;9(10):e1003678.

Lucas-Hourani, M, Dauzonne, D, Munier-Lehmann, H, Khiar, S, Nisole, S, Dairou, J, et al. Original Chemical Series of Pyrimidine Biosynthesis Inhibitors That Boost the Antiviral Interferon Response. Antimicrob Agents Chemother 2017;61(10).

Lucas-Hourani, M, Munier-Lehmann, H, El Mazouni, F, Malmquist, NA, Harpon, J, Coutant, EP, et al. Original 2-(3-Alkoxy-1H-pyrazol-1-yl)azines Inhibitors of Human Dihydroorotate Dehydrogenase (DHODH). J Med Chem 2015;58(14):5579-5598.

Luthra, P, Naidoo, J, Pietzsch, CA, De, S, Khadka, S, Anantpadma, M, et al. Inhibiting pyrimidine biosynthesis impairs Ebola virus replication through depletion of nucleoside pools and activation of innate immune responses. Antiviral Res 2018;158:288-302.

Ma, LL, Wu, ZT, Wang, L, Zhang, XF, Wang, J, Chen, C, et al. Inhibition of hepatic cytochrome P450 enzymes and sodium/bile acid cotransporter exacerbates leflunomide-induced hepatotoxicity. Acta Pharmacol Sin 2016;37(3):415-424.

Maronpot, RR, Yoshizawa, K, Nyska, A, Harada, T, Flake, G, Mueller, G, et al. Hepatic enzyme induction: histopathology. Toxicol Pathol 2010;38(5):776-795.

Maroun, J, Ruckdeschel, J, Natale, R, Morgan, R, Dallaire, B, Sisk, R, et al. Multicenter phase II study of brequinar sodium in patients with advanced lung cancer. Cancer Chemother Pharmacol 1993;32(1):64-66.

Mei-jiao, G, Shi-fang, L, Yan-yan, C, Jun-jun, S, Yue-feng, S, Ting-ting, R, et al. Antiviral effects of selected IMPDH and DHODH inhibitors against foot and mouth disease virus. Biomedicine & Pharmacotherapy 2019;118:109305.

Moore, M, Maroun, J, Robert, F, Natale, R, Neidhart, J, Dallaire, B, et al. Multicenter phase II study of brequinar sodium in patients with advanced gastrointestinal cancer. Invest New Drugs 1993;11(1):61-65.

Munier-Lehmann, H, Lucas-Hourani, M, Guillou, S, Helynck, O, Zanghi, G, Noel, A, et al. Original 2-(3-alkoxy-1H-pyrazol-1-yl)pyrimidine derivatives as inhibitors of human dihydroorotate dehydrogenase (DHODH). J Med Chem 2015;58(2):860-877.

Munier-Lehmann, H, Vidalain, PO, Tangy, F and Janin, YL. On dihydroorotate dehydrogenases and their inhibitors and uses. J Med Chem 2013;56(8):3148-3167.

Ortiz-Riano, E, Ngo, N, Devito, S, Eggink, D, Munger, J, Shaw, ML, et al. Inhibition of arenavirus by A3, a pyrimidine biosynthesis inhibitor. J Virol 2014;88(2):878-889.

Plotkin, SR, Stemmer-Rachamimov, AO, Barker, FG, 2nd, Halpin, C, Padera, TP, Tyrrell, A, et al. Hearing improvement after bevacizumab in patients with neurofibromatosis type 2. N Engl J Med 2009;361(4):358-367.

Raimondo, F, Azzaro, MP, Palumbo, GA, Bagnato, S, Stagno, F, Giustolisi, GM, et al. Elevated vascular endothelial growth factor (VEGF) serum levels in idiopathic myelofibrosis. Leukemia 2001;15(6):976-980.

Russell, B, Moss, C, George, G, Santaolalla, A, Cope, A, Papa, S, et al. Associations between immune-suppressive and stimulating drugs and novel COVID-19—a systematic review of current evidence. ecancermedicalscience 2020;14:1022.

Shi, Q, Yang, X, Greenhaw, J and Salminen, WF. Hepatic cytochrome P450s attenuate the cytotoxicity induced by leflunomide and its active metabolite A77 1726 in primary cultured rat hepatocytes. Toxicol Sci 2011;122(2):579-586.

Shi, Y, Wang, Y, Shao, C, Huang, J, Gan, J, Huang, X, et al. COVID-19 infection: the perspectives on immune responses. Cell Death Differ 2020.

Sykes, DB. The emergence of dihydroorotate dehydrogenase (DHODH) as a therapeutic target in acute myeloid leukemia. Expert Opin Ther Targets 2018;22(11):893-898.

Urba, S, Doroshow, J, Cripps, C, Robert, F, Velez-Garcia, E, Dallaire, B, et al. Multicenter phase II trial of brequinar sodium in patients with advanced squamous-cell carcinoma of the head and neck. Cancer Chemother Pharmacol 1992;31(2):167-169.

Wang, C, Horby, PW, Hayden, FG and Gao, GF. A novel coronavirus outbreak of global health concern. Lancet 2020a;395(10223):470-473.

Wang, D, Hu, B, Hu, C, Zhu, F, Liu, X, Zhang, J, et al. Clinical Characteristics of 138 Hospitalized Patients With 2019 Novel Coronavirus-Infected Pneumonia in Wuhan, China. Jama 2020b.

Wang, QY, Bushell, S, Qing, M, Xu, HY, Bonavia, A, Nunes, S, et al. Inhibition of dengue virus through suppression of host pyrimidine biosynthesis. J Virol 2011;85(13):6548-6556.

Wong, CK, Lam, CW, Wu, AK, Ip, WK, Lee, NL, Chan, IH, et al. Plasma inflammatory cytokines and chemokines in severe acute respiratory syndrome. Clin Exp Immunol 2004;136(1):95-103.

Wu, A, Peng, Y, Huang, B, Ding, X, Wang, X, Niu, P, et al. Genome Composition and Divergence of the Novel Coronavirus (2019-nCoV) Originating in China. Cell Host Microbe 2020;27(3):325-328.

Xiong, R, Zhang, L, Li, S, Sun, Y, Ding, M, Wang, Y, et al. Novel and potent inhibitors targeting DHODH, a rate-limiting enzyme in de novo pyrimidine biosynthesis, are broad-spectrum antiviral against RNA viruses including newly emerged coronavirus SARS-CoV-2. bioRxiv 2020:2020.2003.2011.983056.

Xu, Z, Shi, L, Wang, Y, Zhang, J, Huang, L, Zhang, C, et al. Pathological findings of COVID-19 associated with acute respiratory distress syndrome. Lancet Respir Med 2020.

Yang, CF, Gopula, B, Liang, JJ, Li, JK, Chen, SY, Lee, YL, et al. Novel AR-12 derivatives, P12-23 and P12-34, inhibit flavivirus replication by blocking host de novo pyrimidine biosynthesis. Emerg Microbes Infect 2018;7(1):187.

Yokota, S. [Influenza-associated encephalopathy--pathophysiology and disease mechanisms]. Nihon Rinsho 2003;61(11):1953-1958.

Yoshikawa, N, Yoshikawa, T, Hill, T, Huang, C, Watts, DM, Makino, S, et al. Differential virological and immunological outcome of severe acute respiratory syndrome coronavirus infection in susceptible and resistant transgenic mice expressing human angiotensin-converting enzyme 2. J Virol 2009;83(11):5451-5465.

Yuen, KY and Wong, SS. Human infection by avian influenza A H5N1. Hong Kong Med J 2005;11(3):189-199.

Zhang, Y, Li, J, Zhan, Y, Wu, L, Yu, X, Zhang, W, et al. Analysis of serum cytokines in patients with severe acute respiratory syndrome. Infect Immun 2004;72(8):4410-4415.

We claim:

1. A method of treating coronavirus 2019 disease (COVID-19) caused by severe respiratory syndrome corona virus 2 (SARS-CoV-2) by administering an effective amount of 4-chlorophenyl (S)-6-chloro-1-(4-methoxyphenyl)-1,3,4,9-tetrahydro-2H-pyrido[3,4-b]indole-2-carboxylate (PTC299), having the structure: 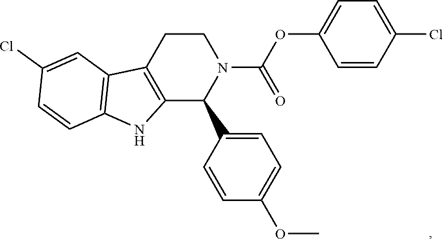
or a pharmaceutically acceptable salt thereof to a patient in need thereof.

2. The method of claim 1, wherein administration of the effective amount of PTC299 results in inhibition of cytokine storms in the patient.

3. The method of claim 1, wherein administration of the effective amount of PTC299 results in inhibition of replication of the SARS-CoV-2.

4. The method of claim 1, wherein the PTC299 is administered in combination with at least one other antiviral agent.

5. The method of claim 4, wherein the at least one other antiviral agent is remdesivir.

6. A method of inhibiting or reducing replication of SARS-CoV-2 in a mammalian cell infected therewith by contacting the cell with an effective amount of 4-chlorophenyl (S)-6-chloro-1-(4-methoxyphenyl)-1,3,4,9-tetrahydro-2H-pyrido[3,4-b]indole-2-carboxylate (PTC299), having the structure: 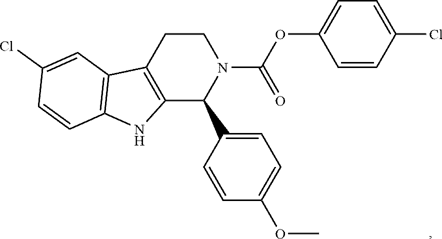
or a pharmaceutically acceptable salt thereof.

7. The method of claim 6, wherein the PTC299 is administered in combination with at least one other antiviral agent.

8. The method of claim 7, wherein the at least one other antiviral agent is remdesivir.
